(19) United States
(12) Patent Application Publication
(10) Pub. No.: US 2022/0036993 A1
(43) Pub. Date: Feb. 3, 2022

(54) PEXIDARTINIB RISK EVALUATION AND MITIGATION STRATEGY (REMS)

(71) Applicant: DAIICHI SANKYO COMPANY, LIMITED, Tokyo (JP)

(72) Inventors: Dale Edward SHUSTER, South Orange, NJ (US); Hamim ZAHIR, Edison, NJ (US); Ophelia Qiping YIN, Westfield, NJ (US); Leonidas Daniel DROGARIS, Holmdel, NJ (US); Vikram Jeet DEV, Hockessin, DE (US); Laurie DELEVE, Los Angeles, CA (US); James Hendricks LEWIS, Potomac, MD (US)

(73) Assignee: DAIICHI SANKYO COMPANY, LIMITED, Tokyo (JP)

(21) Appl. No.: 17/390,217

(22) Filed: Jul. 30, 2021

Related U.S. Application Data
(60) Provisional application No. 63/059,818, filed on Jul. 31, 2020.

(51) Int. Cl. G16H 20/17; A61B 5/145; A61B 5/00; G16H 10/20; G16H 50/30; A61K 31/444

(52) U.S. Cl. CPC G16H 20/17; A61B 5/14546; A61K 31/444; G16H 10/20; G16H 50/30; A61B 5/4848

(57) ABSTRACT

The present disclosure relates generally to methods for using pexidartinib and related Risk Evaluation and Mitigation Strategy (REMS), while reducing the occurrence of hepatotoxic adverse events.

### RELATED APPLICATIONS

This application claims the benefit of U.S. Provisional Application No. 63/059,818, filed Jul. 31, 2020, the disclosure of which is incorporated herein by reference in its entirety.

### FIELD

The present disclosure relates generally to methods of administering pexidartinib, named 5-[(5-chloro-1H-pyrrolo[2,3-b]pyridin-3-yl)methyl]-N-{[6-(trifluoromethyl)pyridin-3-yl]methyl}pyridin-2-amine, or a pharmaceutically acceptable salt thereof, while mitigating certain adverse events.

### BACKGROUND

Pexidartinib in the form of pexidartinib hydrochloride (hereinafter referred to as simply pexidartinib), is an approved drug in the United States and is effective in the treatment of c-Kit and/or c-Fms and/or Flt3 mediated diseases or conditions. Pexidartinib is currently prescribed as Turalio®. Pexidartinib, and preparation thereof, is described in U.S. Pat. No. 7,893,075 as compound P-0181. Certain polymorphs of pexidartinib are described in U.S. Pat. No. 9,802,932. Certain formulations of pexidartinib are described in U.S. Pat. No. 10,435,404. Because the use of pexidartinib by patients may potentially result in hepatotoxicity as a side effect, there is a need for protocols that allow for the safe administration of pexidartinib.

### SUMMARY

The present disclosure, in one embodiment, provides methods for using pexidartinib, or a pharmaceutically acceptable salt thereof, while mitigating the occurrence of hepatotoxic adverse events.

In a first aspect, provided herein is a method of administering pexidartinib, or a pharmaceutically acceptable salt thereof, to a patient while avoiding serious and potentially fatal liver injury comprising:
a) administering to the patient a dose of 400 mg equivalent of pexidartinib twice daily;
b) monitoring the patient by measuring the patient's liver function; and
c) reducing the dose or permanently discontinuing administration based on the patient's liver function.

In another aspect, provided herein is a method for administering pexidartinib, or a pharmaceutically acceptable salt thereof, to a patient while minimizing a risk of serious and potentially fatal liver injury comprising:
a) registering one or more healthcare providers in a healthcare provider database in a non-transitory computer readable storage medium, wherein the one or more healthcare providers have completed a knowledge assessment regarding pexidartinib;
b) requiring the one or more healthcare providers to assess the patient's baseline liver function;
c) registering the patient in a database, wherein the patient has reviewed a patient guide, and has had a baseline liver function assessed; and
d) during treatment, re-assessing the patient's liver function and modifying a dose of pexidartinib, or a pharmaceutically acceptable salt thereof, as needed.

In some embodiments, the method further comprises a step of administering the modified dose of pexidartinib, or a pharmaceutically acceptable salt thereof, to the patient.

In some embodiments, provided herein is a method for administering pexidartinib, or a pharmaceutically acceptable salt thereof, to a patient while minimizing a risk of serious and potentially fatal liver injury, the method comprising:
a) registering one or more healthcare providers in a healthcare provider database in a non-transitory computer readable storage medium, wherein the one or more healthcare providers have completed a knowledge assessment regarding pexidartinib;
b) requiring the one or more healthcare providers to assess the patient's baseline liver function;
c) registering the patient in a database, wherein the patient has reviewed a patient guide, and has had a baseline liver function assessed;
d) during treatment, re-assessing the patient's liver functions and modifying a dose of pexidartinib, or a pharmaceutically acceptable salt thereof, as needed; and
e) administering to the patient the modified dose of pexidartinib, or a pharmaceutically acceptable salt thereof.

In one aspect, provided herein is a method of treating a patient with pexidartinib, or a pharmaceutically acceptable salt thereof, while avoiding serious and potentially fatal liver injury, wherein the patient having symptomatic tenosynovial giant cell tumor (TGCT), the method comprising:
a) administering to the patient a dose of 400 mg equivalent of pexidartinib twice daily;
b) monitoring the patient by measuring the patient's liver function; and
c) reducing the dose or permanently discontinuing administration based on the patient's liver function.

In one aspect provided herein is a method for treating a patient with pexidartinib, or a pharmaceutically acceptable salt thereof, while minimizing a risk of serious and potentially fatal liver injury, wherein the patient having symptomatic tenosynovial giant cell tumor (TGCT), the method comprising:
a) administering to the patient a dose of 400 mg equivalent of pexidartinib twice daily;
b) registering one or more healthcare providers in a healthcare provider database in a non-transitory computer readable storage medium, wherein the one or more healthcare providers have completed a knowledge assessment regarding pexidartinib;
c) requiring the one or more healthcare providers to assess the patient's baseline liver function;
d) registering the patient in a database, wherein the patient has reviewed a patient guide and has had a baseline liver function assessed; and
e) during treatment, re-assessing the patient's liver function and reducing the dose or permanently discontinuing administration based on the patient's liver function.

In some embodiments, the method further comprises a step of administering the modified dose of pexidartinib, or a pharmaceutically acceptable salt thereof, to the patient.

In some embodiments, provided herein is a method for treating a patient with pexidartinib, or a pharmaceutically acceptable salt thereof, while minimizing a risk of serious and potentially fatal liver injury, wherein the patient having symptomatic tenosynovial giant cell tumor (TGCT), the method comprising:
a) administering to the patient a dose of 400 mg equivalent of pexidartinib twice daily;
b) registering one or more healthcare providers in a healthcare provider database in a non-transitory computer readable storage medium, wherein the one or more healthcare providers have completed a knowledge assessment regarding pexidartinib;
c) requiring the one or more healthcare providers to assess the patient's baseline liver function;
d) registering the patient in a database, wherein the patient has reviewed a patient guide and has had a baseline liver function assessed;
e) during treatment, re-assessing the patient's liver function and reducing the dose or permanently discontinuing administration based on the patient's liver function; and
f) administering the modified dose of pexidartinib, or a pharmaceutically acceptable salt thereof, to the patient.

In some embodiments, the method further comprises registering pharmacies who have completed a certification program regarding pexidartinib, or a pharmaceutically acceptable salt thereof, in a pharmacy database, and wherein the pharmacies only dispense pexidartinib, or a pharmaceutically acceptable salt thereof, to patients who are registered in the patient database.

For any method described herein, in some embodiments, the pharmaceutically acceptable salt of pexidartinib is pexidartinib hydrochloride.

For any method described herein, in some embodiments, the patient is being treated for symptomatic tenosynovial giant cell tumor (TGCT).

For any method described herein, in some embodiments, the measuring of the patient's liver function comprises measuring levels of alanine transaminase (ALT), aspartate transaminase (AST), alkaline phosphatase (ALP), gamma-glutamyl transferase (GGT), total bilirubin (TB) and direct bilirubin (DB) or a combination thereof.

For any method described herein, in some embodiments, if the ALT or AST level is greater than 3 to 5 times the corresponding upper limit of normal (ULN), then
a) the administration of pexidartinib, or a pharmaceutically acceptable salt thereof, is withheld and the liver function measurements are conducted weekly; and
b) if the AST and ALT levels are less than or equal to 3 times the corresponding ULN within 4 weeks, the administration of pexidartinib, or a pharmaceutically acceptable salt thereof, is resumed at a reduced dose; or
c) if the AST or ALT level is not less than or equal to 3 times the corresponding ULN in 4 weeks, the administration of pexidartinib, or a pharmaceutically acceptable salt thereof, is permanently discontinued.

For any method described herein, in some embodiments, if the ALT or AST level is greater than 5 to 10 times the corresponding upper limit of normal (ULN), then
a) the administration of pexidartinib, or a pharmaceutically acceptable salt thereof, is withheld and the liver function measurements are conducted twice a week; and
b) if the AST and ALT levels are less than or equal to 3 times the corresponding ULN within 4 weeks, the administration of pexidartinib, or a pharmaceutically acceptable salt thereof, is resumed at a reduced dose; or
c) if the AST or ALT level is not less than or equal to 3 times ULN in 4 weeks, the administration of pexidartinib, or a pharmaceutically acceptable salt thereof, is permanently discontinued.

For any method described herein, in some embodiments, if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then the liver function measurements are conducted weekly until the AST or ALT levels are less than or equal to 3 times the corresponding ULN.

For any method described herein, in some embodiments, if the ALP level is greater than 2 times the corresponding ULN with the GGT greater than 2 times the corresponding ULN, then
a) the administration of pexidartinib, or a pharmaceutically acceptable salt thereof, is permanently discontinued; and
b) the liver function measurements are conducted twice a week until ALP level is less than or equal to 5 times the corresponding ULN, and then the liver function measurements are conducted weekly until ALP level is are less than or equal to 2 times the corresponding ULN.

For any method described herein, in some embodiments, the assessing the patient's liver function comprises measuring levels of total bilirubin (TB), direct bilirubin (DB), or a combination thereof.
For any method described herein, in some embodiments, if TB is greater than the corresponding ULN to less than 2 times the corresponding ULN or DB is greater than the corresponding ULN and less than 1.5 times the corresponding ULN, then
a) the administration of pexidartinib, or a pharmaceutically acceptable salt thereof, is withheld and liver function measurements are conducted twice a week; and
b) if an alternate cause for the increased bilirubin is confirmed and the TB or DB level is less than the corresponding ULN within 4 weeks, the administration of pexidartinib, or a pharmaceutically acceptable salt thereof, is resumed at a reduced dose; or
c) if the TB or DB level is not less than the corresponding ULN in 4 weeks, the administration of pexidartinib, or a pharmaceutically acceptable salt thereof, is permanently discontinued.

For any method described herein, in some embodiments, if the TB level is greater than or equal to 2 times the corresponding ULN or DB is greater than 1.5 times the corresponding ULN, then
a) the administration of pexidartinib, or a pharmaceutically acceptable salt thereof, is permanently discontinued; and
b) the liver function measurements are conducted twice a week until the TB or DB levels is less than or equal to the corresponding ULN.

For any method described herein, in some embodiments, pexidartinib, or a pharmaceutically acceptable salt thereof, is not co-administered with a CYP3A inhibitor, a UDP-glucuronosyltransferase (UGT) inhibitor, or a combination thereof.

For any method described herein, in some embodiments, pexidartinib, or a pharmaceutically acceptable salt thereof, is administered at a reduced dose if co-administered with a CYP3A inhibitor, a UDP-glucuronosyltransferase (UGT) inhibitor, or a combination thereof.

For any method described herein, in some embodiments, pexidartinib, or a pharmaceutically acceptable salt thereof, is not co-administered with a proton pump inhibitor.

For any method described herein, in some embodiments, pexidartinib, or a pharmaceutically acceptable salt thereof, is administered at a reduced dose in patients with mild to severe renal impairment as defined by creatinine clearance [CLcr] 15 to 89 mL/min estimated by Cockcroft-Gault using actual body weight.

For any method described herein, in some embodiments, a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

In an interrelated aspect, at least one computing device receives data encapsulating a request to fill a prescription for pexidartinib, or a pharmaceutically acceptable salt thereof, for a patient. The request can specify information relevant to the prescription including a pharmacy initiating the request and an identification of the patient. In response to the request, a server is polled with the pharmacy identification and the patient identification to determine whether (i) the pharmacy is registered and pre-approved to fill prescriptions for pexidartinib, or a pharmaceutically acceptable salt thereof, (ii) the patient executed a consent form or other acknowledgement associated with the administration of pexidartinib, or a pharmaceutically acceptable salt thereof, and (iii) the patient has a patient status form prepared by a prescribing entity transmitted or prepared within a corresponding validity window thereby making such patient status form active. The validity windows can increase in length of days as treatment of the patient with pexidartinib, or a pharmaceutically acceptable salt thereof, progresses. Data responsive to the request can be transmitted approving filling of the prescription by the pharmacy if it is determined that the pharmacy is registered and pre-approved to fill prescriptions for pexidartinib, or a pharmaceutically acceptable salt thereof, the patient has executed the consent form or other acknowledgment, and the patient has an active patient status form. Alternatively, data responsive to the request can be transmitted rejecting filling of the prescription by the pharmacy if it is determined (a) that the pharmacy is not registered and pre-approved to fill prescriptions for pexidartinib, or a pharmaceutically acceptable salt thereof, (b) the patient has executed the form, or (c) the patient does not have an active patient status form.

The patient status form can encapsulates data regarding at least one liver function test performed by the patient prior to and/or after commencement of treatment. Such liver function can be conducted on a frequency that changes based on progression of the patient treatment. For example, tests can be conducted on a frequency of weekly for the first eight weeks of treatment, every two weeks during a third month of treatment, every three months until a first year anniversary of treatment and every six months thereafter.

The validity windows can have a length equal to one month for months 1 to 3 from a benchmark date, the validity windows have a length equal to three months for months 3 to 12 from the benchmark date, and the validity windows have a length equal to six months after month 12 from the benchmark date. The benchmark date can be a date on which the prescription for pexidartinib, or a pharmaceutically acceptable salt thereof, was first shipped, received in the mail, or otherwise obtained (e.g., in-person at a pharmacy) by the patient.

In some cases, a determination can be made that a most recent patient status form for the patient is outside a corresponding validity window but within a pre-specified grace period. In such cases, data responsive to the request can be transmitted approving filling of the prescription by the pharmacy if it is determined that the pharmacy is registered and pre-approved to fill prescriptions for pexidartinib, or a pharmaceutically acceptable salt thereof, and the patient has executed the consent form or other acknowledgment.

The grace period can vary and, in some implementations, is twenty (20) days.

In some cases, it is determined that a most recent patient status form for the patient is outside a corresponding validity window and outside a pre-specified grace period. In such cases, the patient can be marked as inactive at the server in response to the determination that a most recent patient status form for the patient is outside the corresponding validity window and outside the pre-specified grace period. In addition or in the alternative, data can be transmitted to the prescribing entity indicating that the patient is overdue for a patient status form and requesting the prescribing entity to submit an updated patient status form.

The server can additionally be polled with an identification of a prescribing entity such that the request to fill the prescription is approved if the server indicates that the prescribing entity is authorized to fill the prescription and the request to fill the prescription is rejected if the server indicates that the prescribing entity is not authorized to fill the prescription.

Data can transmitted responsive to the request modifying a dosage and/or dosage frequency based on the patient status form and/or liver function assessment.

The server can take various forms and can be a risk evaluation and mitigation strategies (REMS) server having an associated database storing patient, prescribing entity, and pharmacy information.

Data can be transmitted to the REMS server to register healthcare providers in the database who have completed a knowledge assessment regarding pexidartinib. As used herein, the term “knowledge assessment regarding pexidartinib” includes a knowledge assessment of pexidartinib, or a pharmaceutically acceptable salt thereof. Lab work data characterizing liver function of the patient during treatment with pexidartinib, or a pharmaceutically acceptable salt thereof, can be transmitted to the REMS server for storage in the database. In addition, data can be transmitted to the REMS server to register patients in the database. Such patient registration data can include or otherwise characterize an acknowledgment that a patient guide has been reviewed. Further, data can be transmitted to the REMS server to register pharmacies in the database who have completed a certification program regarding pexidartinib, or a pharmaceutically acceptable salt thereof, such that the pharmacies only dispense pexidartinib, or a pharmaceutically acceptable salt thereof, to patients who are registered in the patient database.

Computer program products are also described that comprise computer executable instructions permanently stored on computer readable media, which, when executed by one or more computing devices, causes the computing device(s) to perform operations herein. Similarly, computer systems are also described that may include a processor and a memory coupled to the processor. The memory may temporarily or permanently store one or more programs that cause the processor to perform one or more of the operations described herein. In addition, aspects of methods can be implemented by one or more data processors either within a single computing device or distributed among two or more computing devices.

The subject matter described herein provides many advantages. For example, the current subject matter provides an enhanced, computer-implemented risk evaluation and mitigation strategies which ensure appropriate patient care when prescribing pexidartinib, or a pharmaceutically acceptable salt thereof.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a computer system diagram illustrating data exchange among computing devices in connection with the administration of pexidartinib, or a pharmaceutically acceptable salt thereof;

FIG. 2 is a process flow diagram illustrating aspects associated with the administration of pexidartinib, or a pharmaceutically acceptable salt thereof; and

FIG. 3 is a diagram of a computing device for implementing aspects of the current subject matter.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

As used herein, the terms “treat”, “treating”, “therapy”, “therapies”, and like terms refer to the administration of material, e.g., any one or more compound(s) as described herein in an amount effective to prevent, alleviate, or ameliorate one or more symptoms of a disease or condition, i.e., indication, and/or to prolong the survival of the subject being treated.

Pexidartinib is an inhibitor of Fms, Kit and Flt3 protein kinases. The kinase assays that can measure the IC₅₀ values for these targets are described in US Publication Nos. US 2007/0032519, US 2009/0076046 and US 2011/0112127. Pexidartinib has IC₅₀ values of less than 0.05 μM for each of these three kinase targets.

As used herein, the term “Fms and/or Kit and/or Flt3 protein kinase mediated disease or condition” refers to a disease or condition in which the biological function of a Fms protein kinase, including any mutation thereof, a Kit protein kinase, including any mutation thereof, a Flt3 protein kinase, including any mutation thereof or both a Fms and Kit protein kinase, including any mutations thereof, affects the development, course, and/or symptoms of the disease or condition, and/or in which modulation of the Fms and/or Kit and/or Flt3 protein kinase alters the development, course, and/or symptoms of the disease or condition. A Fms and/or Kit and/or Flt3 protein kinase mediated disease or condition includes a disease or condition for which modulation provides a therapeutic benefit, e.g. wherein treatment with Fms and/or Kit and/or Flt3 protein kinase inhibitor(s), including one or more solid, crystalline or polymorphs of pexidartinib or solid or crystalline forms of pexidartinib as described herein, or a composition thereof as described herein, and optionally in combination with another therapeutic agent or therapy as described herein provides a therapeutic benefit to the subject having, or at risk of the disease or condition.

As used herein, the term “modulating” or “modulate” refers to an effect of altering a biological activity, especially a biological activity associated with a particular biomolecule such as a protein kinase. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme.

As used herein, the term “composition” refers to a pharmaceutical preparation suitable for administration to an intended subject for therapeutic purposes that contains at least one pharmaceutically active compound, including any solid form thereof. The composition may include at least one pharmaceutically acceptable component to provide an improved formulation of the compound, such as a suitable carrier or excipient.

As used herein, the term “patient” refers to a living organism that is treated with compounds as described herein, including, but not limited to, any mammal, such as a human, other primates, sports animals, animals of commercial interest such as cattle, farm animals such as horses, or pets such as dogs and cats. “Patient” may be used interchangeably with “subject” herein.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

As used herein, the terms “pexidartinib treatment” or “pexidartinib therapy” refer to a treatment with pexidartinib or a pharmaceutically acceptable salt thereof, in some embodiments, a treatment with pexidartinib, and still further embodiments, a treatment with pexidartinib hydrochloride.

The term “therapeutically effective amount” or “effective amount” of a compound described herein means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition mediated by c-Kit and/or c-Fms, including, but not limited to, symptomatic tenosynovial giant cell tumor (TGCT). The therapeutically effective amount may vary depending on the subject, and disease or condition being treated, the weight and age of the subject, the severity of the disease or condition, and the manner of administering, which can readily be determined by one or ordinary skill in the art.

As used herein, the terms “liver injury” “liver damage” or “hepatotoxicity” refer histological or biochemical dysfunction of liver. The term “liver function” refers to one or more physiological functions performed by the liver. Liver function can be analyzed by conventional assays known to one of skill in the art, including, but not limited to, measuring levels of alanine transaminase (ALT), aspartate transaminase (AST), alkaline phosphatase (ALP), gamma-glutamyl transferase (GGT), or a combination thereof. Liver function may also be assessed by measuring levels of total bilirubin (TB), direct bilirubin (DB), or a combination thereof. In some embodiments, liver injury is defined as ALT or AST greater than or equal to 3 times the corresponding upper limit of normal (ULN) with total bilirubin greater than or equal to 2 times the corresponding ULN.

For the liver function measurements disclosed herein, for example, measurement of the levels of alanine transaminase (ALT), aspartate transaminase (AST), alkaline phosphatase (ALP), gamma-glutamyl transferase (GGT), total bilirubin (TB), and bilirubin (DB), and the like, the comparative values “1.5 times,” “2 times,” “3 times,” “5 times,” “10 times,” etc., will be understood to mean within an acceptable error range for the particular value as determined by one of ordinary skill in the art, which will depend in part on how the value is measured or determined, i.e., the limitations of the measurement system. The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for tablets.

The term “pharmaceutically acceptable salt” as used herein refers to non-toxic acid addition salts of pexidartinib. In some embodiments, the pharmaceutically acceptable salt include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid. Each of the above-mentioned salts is prepared by methods known to one of skill in the art.

As described herein, in some embodiments, pexidartinib refers to both the pexidartinib free base and a pharmaceutically acceptable salt of pexidartinib (e.g., pexidartinib hydrochloride), including crystalline forms of pexidartinib free base and pharmaceutically acceptable salts (e.g. crystalline forms of pexidartinib hydrochloride).

Pexidartinib, or more specifically pexidartinib hydrochloride, has a chemical name of 5-[(5-chloro-1H-pyrrolo[2,3-b]pyridin-3-yl)methyl]-N-[6-(trifluoromethyl)pyridin-3-yl]methyl pyridin-2-amine HCl salt and has the following structure:

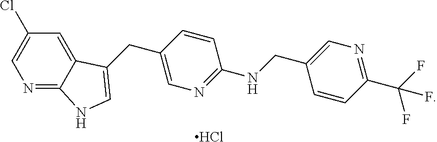 [SMILES: FC(F)(F)c1ccc(CNc2ccc(Cc3c[nH]c4ncc(Cl)cc34)cn2)cn1 ; Cl]

The molecular formula for pexidartinib hydrochloride is C₂₀H₁₅ClF₃N₅.HCl. The molecular weight is 454.28 for the hydrochloride salt and 417.81 for the free base.

One of skill in the art would understand that 200 mg dose of pexidartinib is equivalent to 217.5 mg pexidartinib hydrochloride. One of skill in the art would also understand that the amount of a pharmaceutically acceptable salt of pexidartinib is provided in sufficient amount to provide the equivalent dose of pexidartinib after the acid moiety is removed from the salt (e.g. 217.5 mg pexidartinib hydrochloride provides 200 mg pexidartinib free base). As used herein, the language “equivalent of pexidartinib,” “equivalent dose of pexidartinib,” “pexidartinib dose equivalent,” and the like, refers to a dose of pexidartinib, a pexidartinib salt (e.g., pexidartinib hydrochloride), or a crystalline form of a pexidartinib salt, which dose is equivalent to a 200 mg dose of the pexidartinib free base.

In some embodiments, a crystalline form of pexidartinib hydrochloride is pexidartinib Form C, which is characterized by an X-ray powder diffractogram comprising peaks (±0.2°) at 7.3, 23.3 and 28.2° 20 as determined on a diffractometer using Cu-Kα radiation. Pexidartinib Form C is further characterized by: i) peaks at 16.6 and 20.9° 20±0.2° as determined on a diffractometer using Cu-Kα radiation; and/or ii) a differential scanning calorimetry (DSC) thermogram comprising an endotherm at about 234° C. Pexidartinib is useful for treatment of subjects having, or at risk of a c-Kit and/or c-Fms mediated disease and is currently prescribed for patients having symptomatic tenosynovial giant cell tumor (TGCT).

### Administration of Pexidartinib with Control of Potential Liver Toxicity

Pexidartinib is a drug for the treatment of c-Kit and/or c-Fms mediated diseases, including, and not limited to, treatment of symptomatic tenosynovial giant cell tumor (TGCT) when associated with severe morbidity and/or functional limitations and when surgery is not expected to improve morbidity or functional limitations. The benefits of any drug administration have to be typically balanced versus the possibility of adverse side effects. For instance, some drugs interact with liver enzymes and may not be suitable for co-administration with other drugs that may also impede the function of liver enzymes. Where a drug affects liver function, it may be desirable to administer the drug at reduced doses to minimize liver toxicity. In other instances, certain drugs have therapeutic windows that vary between individuals and the optimum therapeutic dose for any individual may need to be titrated under the guidance of a qualified physician.

It is discovered that the use of pexidartinib is associated with potential liver toxicities. In some embodiments, the methods described herein comprise administering pexidartinib to a patient in need thereof while reducing the occurrence of hepatotoxic adverse events. Therefore, while a patient is being treated with pexidartinib, it is helpful to monitor the patient's liver function. In some embodiments, the liver function is assessed by measuring levels of alanine transaminase (ALT), aspartate transaminase (AST), alkaline phosphatase (ALP), gamma-glutamyl transferase (GGT), or a combination thereof. Liver function may also be assessed by measuring levels of total bilirubin (TB), direct bilirubin (DB), or a combination thereof. Depending on the assessment of the liver function, the dose or dosing schedule of pexidartinib can be adjusted as shown in Tables 1-3 herein, and as further described herein. It is also helpful to monitor any other medications (e.g., medications that may inhibit liver enzymes such as the cytochrome P450 enzymes) that have been co-prescribed for the patient. In some instances it may be helpful to change the patient's co-administered medication to avoid any drug-drug interactions and/or cross reactions. By way of example only, in some embodiments, where a patient is being treated with proton pump inhibitors while being prescribed pexidartinib, it may be helpful to treat the patient with histamine-2 receptor antagonists or antacids instead of proton pump inhibitors. In certain instances, if a co-administered medication for a patient cannot be changed while the patient is also being treated with pexidartinib and the co-administered treatment is likely to impact liver function, it is helpful to administer pexidartinib at a reduced dose to minimize any drug-related liver toxicity in the patient.

Patients that may benefit from administration of pexidartinib, include those that suffer from, or are at risk of developing, a Fms and/or Kit and/or Flt3 protein kinase mediated disease or condition.

In some embodiments, the methods described herein involve administering to a patient having, or at risk of developing, a disease or condition mediated by Fms and/or Kit and/or Flt3 protein kinase, an effective amount of pexidartinib while reducing hepatotoxic side effects as described herein.

Examples of diseases and condition mediated by Fms and Kit, without limitation, include rheumatoid arthritis, osteoarthritis, psoriatic arthritis, psoriasis, dermatitis, allergy, anaphylaxis, asthma, allergic rhinitis, ankylosing spondylitis, polymyositis, dermatomyositis, systemic sclerosis, juvenile idiopathic arthritis, polymyalgia rheumatica, Sjogren's disease, Langerhan's cell histiocytosis, Still's disease, inflammatory bowel disease, ulcerative colitis, Crohn's disease, systemic lupus erythematosis, immune thrombocytopenic purpura, myelopreparation for autologous transplantation, transplant rejection, chronic obstructive pulmonary disease, emphysema, Kawasaki's Disease, hemophagocytic syndrome, multicentric reticulohistiocytosis, hypereosinophilia, and urticaria type I diabetes, type II diabetes, insulin resistance, hyperglycemia, obesity, and lipolysis, osteoporosis, osteodystrophy, increased risk of fracture, Paget's disease, hypercalcemia, infection-mediated osteolysis, and peri-prosthetic or wear-debris-mediated osteolysis, endometriosis, nephritis, glomerulonephritis, interstitial nephritis, Lupus nephritis, tubular necrosis, diabetes-associated renal complications, diabetic nephropathy, renal hypertrophy, systemic sclerosis, multiple sclerosis, Charcot Marie Tooth syndrome, amyotrophic lateral sclerosis, myasthenia gravis, chronic demyelinating polyneuropathy, other demyelinating disorders, stroke, Alzheimer's disease and Parkinson's disease, acute pain, neuropathic pain, inflammatory pain, chronic pain, migraine, multiple myeloma, acute lymphocytic leukemia, acute myeloid leukemia, chronic myeloid leukemia, mast cell tumors, canine mast cell tumors, lung cancer, testicular cancer, pancreatic cancer, prostate cancer, breast cancer, metastatic breast cancer, ovarian cancer, merkel cell carcinoma, carcinomas of the female genital tract, colorectal carcinoma, carcinoma in situ, gastrointestinal stromal tumors, tumor angiogenesis, astrocytoma, neuroblastoma, sarcoma, osteosarcoma, sarcomas of neuroectodermal origin, giant cell tumor of bone, giant cell tumor of tendon sheath, pigmented villonodular synovitis, melanoma, glioblastoma, glioblastoma multiforme, glioma, other tumors of the central nervous system, neurofibromatosis (including Schwann cell neoplasia associated with neurofibromatosis), brain metastases, mastocytosis, metastasis of tumors to other tissues, osteolytic bone metastases, and other chronic myeloproliferative diseases such as myelofibrosis, collagen vascular disease, polyarteritis nodosa, Behcet's disease, sarcoidosis, familiar Mediterranean fever, Churg-Strauss vasculitis, temporal arteritis, giant cell arteritis, Takayasu's arteritis, uveitis, scleritis, retinitis, age related macular degeneration, choroidal neovascularization, diabetic retinopathy, cherubism, neurofibromatosis, infections associated with human immunodeficiency virus, hepatitis B virus, hepatitis C virus, human granulocytic anaplasmosis, Gaucher's disease, Fabry's disease, Niemann-Pick disease, liver cirrhosis, gastroesophageal reflux disease, esophagitis, and gastrointestinal tract ulcers, pulmonary fibrosis, acute lung injury, bypass surgery, vascular surgery, and vascular grafts, atherosclerosis, cardiomyopathy, heart failure, and pulmonary arterial hypertension.

Further examples include stem cell ablation and myelopreparation for stem cell transplant, monocytic leukemia, primary progressive multiple sclerosis, complex regional pain syndrome, reflex sympathetic dystrophy, muscular dystrophy, duchenne muscular dystrophy, causalgia, malignant peripheral nerve cell tumors, malignant peripheral nerve sheath tumors, pheochromocytomas cutaneous and plexiform neurofibromas, neuro-inflammations, benign forgetfulness, HIV, binswager type dementia, dementia with lewy bodie, prosencephaly, microencepahy, cerebral palsy, congenital hydrocephalus, tremors, Wilson's disease, vascular dementias/multi infarct dementia, fronto temporal type, pseudo-dementia, papillary thyroid cancer, anaplastic thyroid cancer, medullary thyroid cancer, follicular thyroid cancer, hurthle cell carcinoma, thyroid cancer, ascites, malignant ascites, abdominal dropsy, progressive supranuclear palsy, glaucoma, mesothelioma, salivary gland tumors, mucoepidermoid carcinoma of the salivary gland, acinic cell carcinoma of the salivary gland, and others), gastrointestinal stromal tumors (GIST—which includes, without limitation, 1^{st} line, 2^{nd} line and neoadjuvant GIST), tumors that cause effusions in potential spaces of the body, pleural effusions, pericardial effusions, peritoneal effusions aka ascites, giant cell tumors (GCT), GCT of bone, pigmented villonodular synovitis (PVNS), tenosynovial giant cell tumor (TGCT), giant cell tumor of the tendon sheath (GCT-TS), other sarcomas, tumor angiogenesis and paracrine tumor growth; and tumors that express aberrantly or otherwise Fms, CSF1R, CSF1 or IL-34, or activating mutations or translocations of any of the foregoing, wherein pexidartinib is an inhibitor of Kit. In one embodiment, the condition mediated by Fms and Flt-3 is acute myeloid leukemia.

### Methods of Treatment of Symptomatic Tenosynovial Giant Cell Tumor (TGCT)

In one aspect, provided herein is a method of treating a patient with pexidartinib, or a pharmaceutically acceptable salt thereof, while avoiding serious and potentially fatal liver injury, wherein the patient having symptomatic tenosynovial giant cell tumor (TGCT), the method comprising:
a) administering to the patient a dose of 400 mg equivalent of pexidartinib twice daily;
b) monitoring the patient by measuring the patient's liver function; and
c) reducing the dose or permanently discontinuing administration based on the patient's liver function.

In one aspect provided herein is a method for treating a patient with pexidartinib, or a pharmaceutically acceptable salt thereof, while minimizing a risk of serious and potentially fatal liver injury, wherein the patient having symptomatic tenosynovial giant cell tumor (TGCT), the method comprising:
a) administering to the patient a dose of 400 mg equivalent of pexidartinib twice daily;
b) registering one or more healthcare providers in a healthcare provider database in a non-transitory computer readable storage medium, wherein the one or more healthcare providers have completed a knowledge assessment regarding pexidartinib;
c) requiring the one or more healthcare providers to assess the patient's baseline liver functions;
d) registering the patient in a database, wherein the patient has reviewed a patient guide and has had a baseline liver function assessed; and
e) during treatment, re-assessing a patient's liver function and reducing the dose or permanently discontinuing administration based on the patient's liver function.

In some embodiments, the method further comprises a step of administering the modified dose of pexidartinib, or a pharmaceutically acceptable salt thereof, to the patient.

In some embodiments, the methods described herein comprise administering pexidartinib, or a pharmaceutically acceptable salt thereof, to a patient having symptomatic tenosynovial giant cell tumor while mitigating the occurrence of hepatotoxic adverse events.

In some embodiments, the methods described herein comprise administering pexidartinib, or a pharmaceutically acceptable salt thereof, to adult patients with symptomatic tenosynovial giant cell tumor (TGCT) associated with severe morbidity or functional limitations and not amenable to improvement with surgery.

In some embodiments of the methods provided herein, the measuring of the patient's liver function comprises measuring levels of alanine transaminase (ALT), aspartate transaminase (AST), alkaline phosphatase (ALP), gamma-glutamyl transferase (GGT), total bilirubin (TB), direct bilirubin (DB) or a combination thereof.

In some embodiments of the methods provided herein, if the ALT or AST level is greater than 3 to 5 times the corresponding upper limit of normal (ULN), then the administration of pexidartinib, or a pharmaceutically acceptable salt thereof, is withheld and the liver function measurements are conducted weekly; and
i) if the AST and ALT levels are less than or equal to 3 times the corresponding ULN within 4 weeks, the administration of pexidartinib, or a pharmaceutically acceptable salt thereof, is resumed at a reduced dose; or
ii) if the AST or ALT level is not less than or equal to 3 times the corresponding ULN in 4 weeks, the administration of pexidartinib, or a pharmaceutically acceptable salt thereof, is permanently discontinued.

In some embodiments of the methods provided herein, if the ALT or AST level is greater than 5 to 10 times the corresponding upper limit of normal (ULN), then the administration of pexidartinib, or a pharmaceutically acceptable salt thereof, is withheld and the liver function measurements are conducted twice a week; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times the corresponding ULN in 4 weeks, the administration of pexidartinib, or a pharmaceutically acceptable salt thereof, is permanently discontinued.

In some embodiments of the methods provided herein if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then the liver function measurements are conducted weekly until the AST or ALT levels are less than or equal to 3 times the corresponding ULN.

In some embodiments of the methods provided herein, if the ALP level is greater than 2 times the corresponding ULN and the GGT is greater than 2 times the corresponding ULN, then
a) the administration of pexidartinib, or a pharmaceutically acceptable salt thereof, is permanently discontinued; and
b) the liver function measurements are conducted twice a week until the ALP level is less than or equal to 5 times the corresponding ULN, and then the liver function measurements are conducted weekly until the ALP level is are less than or equal to 2 times the corresponding ULN.

In some embodiments, the measuring the patient's liver function comprises measuring levels of total bilirubin (TB), direct bilirubin (DB), or a combination thereof.

For any method described herein, in some embodiments, the pharmaceutically acceptable salt of pexidartinib is pexidartinib hydrochloride.

In some embodiments, the methods provided herein comprise administering pexidartinib, or a pharmaceutically acceptable salt thereof, at a reduced dose in patients with mild to severe renal impairment as defined by creatinine clearance [CLcr] 15 to 89 mL/min estimated by Cockcroft-Gault using actual body weight. In some embodiments, a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

In some embodiments, pexidartinib, or a pharmaceutically acceptable salt thereof, is administered to a patient in need thereof on an empty stomach, at least 1 hour before or 2 hours after a meal or snack.

Pexidartinib, or a pharmaceutically acceptable salt thereof, may be administered in the form of pharmaceutical compositions that include pexidartinib, or a pharmaceutically acceptable salt thereof, and a pharmaceutically acceptable vehicle. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses, such as orally.

Oral administration may be via, for example, capsule or enteric coated tablets. In making the pharmaceutical compositions that include at least one compound described herein or a pharmaceutically acceptable salt, tautomer, stereoisomer, mixture of stereoisomers, prodrug, or deuterated analog thereof, the active ingredient, e.g., pexidartinib hydrochloride, is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In some embodiments, a composition of pexidartinib comprises: 40% to 60% w/w of pexidartinib hydrochloride wherein pexidartinib hydrochloride is a crystalline HCl salt characterized by an X-ray powder diffractogram comprising peaks (±0.2°) at 7.3, 23.3 and 28.2 °2θ as determined on a diffractometer using Cu-Kα radiation; 20% to 35% w/w of a poloxamer; 10% to 22% w/w of an excipient; 1% to 5% w/w of a disintegrant; and 0.5% to 3% w/w of a lubricant. In some embodiments, the diffractogram further comprises peaks at 16.6 and 20.9 °2θ±0.2°. In some embodiments, the excipient is mannitol; the disintegrant is crospovidone; and the lubricant is magnesium stearate. In some embodiments, the crospovidone is Polyplasdone® Ultra or Polyplasdone® Ultra-10. In some embodiments, the poloxamer is poloxamer 407. In some embodiments, poloxamer is poloxamer 407; the excipient is mannitol; the disintegrant is crospovidone; and the lubricant is magnesium stearate. In some embodiments, the composition is a capsule form where the capsule comprises hypromellose. In some embodiments, the composition comprises 51.2% w/w (±3%) of pexidartinib; 27.6% w/w (±3%) of poloxamer 407; 16.8% (±3%) w/w of mannitol; 3% w/w (±1%) of crospovidone; and 1.5% (±1%) w/w of magnesium stearate.

When administered orally, the total daily dosage for a human subject may be between 1 mg and 1,000 mg, between about 400 to 800 mg/day, or between about 400-600 mg/day.

Pexidartinib, or a pharmaceutically acceptable salt thereof, or compositions thereof may be administered once, twice, three, or four times daily, using any suitable mode described herein. Also, administration or treatment with pexidartinib, or a pharmaceutically acceptable salt thereof, may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In some embodiments, pexidartinib, or a pharmaceutically acceptable salt thereof, is administered at a suitable dose and frequency as determined by a physician, and in combination with liver function testing for the patient. A suitable dose may be a daily dose ranging from about 100 mg equivalent of pexidartinib per day to about 1 g per day, about 200 mg equivalent of pexidartinib to about 800 mg equivalent of pexidartinib per day, about 200 mg equivalent of pexidartinib to about 600 mg equivalent of pexidartinib per day, or about 200 mg equivalent of pexidartinib to about 400 mg equivalent of pexidartinib per day. A typical starting dose is 800 mg equivalent of pexidartinib per day, administered as a split dose of 400 mg equivalent of pexidartinib twice a day (BID). The patient undergoes an initial liver function measurement prior to start of pexidartinib therapy (initial measurement). After initiation of treatment with pexidartinib, the patient undergoes liver function tests at certain intervals of time to determine if the patient's liver enzyme functions are affected by pexidartinib therapy. In some embodiments, the periodic liver function testing allows a physician to establish if the dose of pexidartinib should be maintained or if it should be reduced or if it should be discontinued. In other embodiments, the periodic liver function testing allows a physician to determine if any co-administered drug prescribed for the patient should be changed or discontinued.

Liver function measurements (used herein interchangeably with liver function tests or tests) are blood tests used to help diagnose and monitor liver disease or damage. The tests measure the levels of certain enzymes and proteins in a patient's blood. Some liver function tests measure how well the liver is performing its normal functions of producing protein and/or clearing bilirubin, a waste product in blood. Other liver function tests measure levels of enzymes that liver cells release into the blood in response to damage or disease and/or drug exposure. Any change from the initial measurements for the patient is evaluated to determine if the dose of pexidartinib should be adjusted (e.g., withhold temporarily or reduce the dose, or withhold temporarily and then resume at reduced dose). Liver function test readouts provide numbers that are compared to the corresponding parameter's upper limit of normal (ULN), which is the highest number for that parameter at which the liver is considered to be functioning without any damage or toxicity. The ULN for each liver function measurement is generally known. There may be variations depending on the patient cohort and locale, but the medical professional conducing the measurement can readily provide reference numbers for a patient. In one example, the normal ranges of some typical liver function tests include ALT: 7 to 55 units per liter (U/L), AST: 8 to 48 U/L, ALP: 40 to 129 U/L, albumin: 3.5 to 5.0 grams per deciliter (g/dL); total protein. 6.3 to 7.9 g/dL; bilirubin: 0.1 to 1.2 milligrams per deciliter (mg/dL), GGT: 8 to 61 U/L; LD: 122 to 222 U/L; and PT: 9.4 to 12.5 seconds. In another example, the normal ranges can be ALT: 3-40 iu/l; AST: 3-30 iu/1; ALP: 30-100 μmol/l; GGT: 8-60 u/1; bilirubin: 3-17 μmol/l; albumin: 35-50 g/1; and PT: 10-14 s/l. A liver function measurement for ALT can be compared to the corresponding ULN (i.e., the ULN for ALT). Pexidartinib treatment is typically initiated at a starting dose of 800 mg equivalent of pexidartinib per day and the dose may be modified as needed. In alternate embodiments, pexidartinib treatment may be initiated at a reduced dose (e.g., where a patient suffers from renal impairment).

In some embodiments, if, after pexidartinib treatment is initiated, a patient's ALT and/or AST levels are determined to be greater than 3 to 5 times the corresponding ULN, pexidartinib treatment is suspended, and the patient undergoes liver function tests weekly till the patient's ALT or AST levels are less than 3 times the corresponding ULN. If a reduction in ALT and AST levels to less than 3 times the corresponding ULN is observed by the fourth week of suspended dosing, dosing of pexidartinib may be resumed at a lower dose compared to the starting dose of pexidartinib. If the patient's elevated ALT or AST levels are not decreased to less than 3 times the corresponding ULN by the fourth week, pexidartinib treatment is permanently discontinued for the patient.

In some embodiments, if, after pexidartinib treatment is initiated, a patient's ALT and/or AST levels are determined to be greater than 5 to 10 times the corresponding ULN, pexidartinib treatment is suspended, and the patient undergoes liver function tests twice a week till the patient's ALT or AST levels are less than 3 times the corresponding ULN. If a reduction in ALT and AST levels to less than 3 times the corresponding ULN is observed by the fourth week of suspended dosing, dosing of pexidartinib may be resumed at a lower dose compared to the starting dose of pexidartinib. If the patient's elevated ALT or AST levels are not decreased to less than 3 times the corresponding ULN by the fourth week, pexidartinib treatment is permanently discontinued for the patient.

In other embodiments, if, after pexidartinib treatment is initiated, a patient's ALT and/or AST levels are determined to be greater than 10 times the corresponding ULN, pexidartinib treatment is permanently discontinued for the patient. To avoid any fatal hepatotoxic events, the patient is tested twice a week till the patient's elevated ALT or AST levels are decreased to less than or equal to 5 times the corresponding ULN, then the patient is tested once a week till the patient's elevated ALT or AST levels are decreased to less than or equal to 3 times the corresponding ULN.

In some embodiments, if, after pexidartinib treatment is initiated, a patient's ALP level is determined to be greater than 2 times the corresponding ULN for ALP, and if a patient's GGT level is determined to be greater than 2 times the corresponding ULN for GGT, pexidartinib treatment is permanently discontinued for the patient. To avoid any fatal hepatotoxic events, the patient is tested twice a week till the patient's elevated ALP levels are decreased to less than or equal to 5 times the corresponding ULN, then the patient is tested once a week till the patient's elevated ALP levels are decreased to less than or equal to 2 times the corresponding ULN.

In some embodiments, if, after pexidartinib treatment is initiated, a patient's TB is determined to be greater than the corresponding ULN for TB to less than 2 times the corresponding ULN for TB, pexidartinib treatment is suspended, and the patient undergoes liver function tests twice weekly till the patient's TB levels are less than the corresponding ULN. If an alternate cause for increased bilirubin is confirmed and a reduction in TB levels to less than the corresponding ULN is observed by the fourth week of suspended dosing, dosing of pexidartinib may be resumed at a lower dose compared to the starting dose of pexidartinib. If the patient's elevated TB levels are not decreased to less than the corresponding ULN by the fourth week, pexidartinib treatment is permanently discontinued for the patient.

In some embodiments, if, after pexidartinib treatment is initiated, a patient's DB is determined to be greater than the corresponding ULN for DB and less than 1.5 times the corresponding ULN for DB, pexidartinib treatment is suspended, and the patient undergoes liver function tests twice weekly till the patient's DB levels are less than the corresponding ULN. If an alternate cause for increased bilirubin is confirmed and a reduction in DB levels to less than the corresponding ULN is observed by the fourth week of suspended dosing, dosing of pexidartinib may be resumed at a lower dose compared to the starting dose of pexidartinib. If the patient's elevated DB levels are not decreased to less than the corresponding ULN by the fourth week, pexidartinib treatment is permanently discontinued for the patient.

In some embodiments, if, after pexidartinib treatment is initiated, a patient's TB is determined to be greater than or equal to 2 times the corresponding ULN for TB, pexidartinib treatment is permanently discontinued. To avoid any fatal hepatotoxic events, the patient is tested twice a week till the patient's elevated TB levels are decreased to less than or equal to the corresponding ULN.

In some embodiments, if, after pexidartinib treatment is initiated, a patient's DB is determined to be greater than the corresponding ULN for DB and less than 1.5 times the corresponding ULN for DB, pexidartinib treatment is permanently discontinued. To avoid any fatal hepatotoxic events, the patient is tested twice a week till the patient's elevated DB levels are decreased to less than or equal to the corresponding ULN.

In some embodiments, where a patient suffers from mild to severe renal impairment (creatinine clearance [CLcr] 15 to 89 mL/min estimated by Cockcroft-Gault using actual body weight) pexidartinib treatment may be initiated at a reduced dose (e.g., 200 mg in the morning and 400 mg in the evening) followed by liver function measurements as described herein. The patients daily dosing of pexidartinib may be adjusted (reduced or discontinued) based on liver function measurements.

Accordingly, in some or any of the embodiments described herein, periodic monitoring of a patient's liver function after initiation of pexidartinib therapy (e.g., by measuring ALT and/or AST level, and/or by measuring ALP and GGT levels, and/or by measuring increase in bilirubin) reduces the occurrence of, and mitigates the risk of, fatal hepatotoxicities. Periodic liver function measurements may be undertaken at any suitable interval of time, e.g., each day, every other day, twice a week, once a week, every two weeks, once a month, every six months, once a year, or any other suitable frequency. A co-administered medication may also impact liver function. Accordingly, in some embodiments, administration of pexidartinib with medications that affect liver function should be avoided. Examples of medications that interact with pexidartinib and that may increase the potential of liver injury from pexidartinib include, and are not limited to, CYP3A inhibitors, UDP-glucuronosyltransferase (UGT) inhibitors, and the like. In other embodiments, pexidartinib should be administered at reduced doses if co-administration of medications that may impact liver function cannot be avoided.

In a particular embodiment, the methods described herein comprise administering to the subject an initial daily dose of about 800 mg equivalent of pexidartinib per day described herein and adjusting the dose to avoid serious and potentially fatal liver injury according to the methods described herein. The dosage can be decreased from about 800 mg equivalent of pexidartinib per day to about 600 mg equivalent of pexidartinib per day, about 400 mg equivalent of pexidartinib per day, or about 200 mg equivalent of pexidartinib per day. In some embodiments, the dosing is permanently discontinued. In such embodiments, the dose is adjusted based on liver function assessments as described herein.

In accord with the methods described herein, in some embodiments, a dose reduction of pexidartinib, or a pharmaceutically acceptable salt thereof, is carried out according to Table 1. Starting from a total daily dose of 800 mg equivalent of pexidartinib daily, the first dose reduction comprises reducing the dose of pexidartinib, or a pharmaceutically acceptable salt thereof, to 600 mg equivalent of pexidartinib daily. The second dose reduction comprises reducing the dose of pexidartinib, or a pharmaceutically acceptable salt thereof, to 400 mg equivalent of pexidartinib daily.

TABLE 1

| Dose Reduction | Total Daily | Administration of Total Daily Dose |
|---|---|---|
| First | 600 mg | 200 mg in the morning and 400 mg in |
| | | the evening |
| Second | 400 mg | 200 mg twice daily |

In some embodiments, pexidartinib is permanently discontinued in patients who are unable to tolerate 200 mg orally twice daily.

In some embodiments, a dose reduction of pexidartinib is carried out based on liver function assessments, and according to Table 2. In some embodiments, where there are any adverse reactions observed in a patient that are severe or intolerable, pexidartinib is withheld until there is improvement or resolution of the adverse reactions, and then resumed at a reduced dose upon improvement or resolution of the adverse reactions.

TABLE 2

| Adverse | | |
|---|---|---|
| Reaction | Severity | Pexidartinib Dosage Modifications |
| Increased | Greater than 3 to | Withhold and monitor liver tests weekly. |
| ALT | 5 times ULN | If AST and ALT are less than or equal |
| and/or | | to 3 times ULN within 4 weeks, resume at |
| AST | | reduced dose. |
| | | If AST or ALT is not less than or equal |
| | | to 3 times ULN in 4 weeks, permanently |
| | | discontinue pexidartinib. |
| | Greater than 5 to | Withhold and monitor liver tests twice |
| | 10 times ULN | weekly. |
| | | If AST and ALT are less than or equal |
| | | to 3 times ULN within 4 weeks, resume at |
| | | reduced dose. |
| | | If AST or ALT is not less than or equal |
| | | to 3 times ULN in 4 weeks, permanently |
| | | discontinue pexidartinib. |
| | Greater than 10 | Permanently discontinue pexidartinib. |
| | times ULN | Monitor liver tests twice weekly until |
| | | AST or ALT is less than or equal to 5 |
| | | times ULN, then weekly until less than or |
| | | equal to 3 times ULN. |
| Increased | ALP greater than | Permanently discontinue pexidartinib. |
| ALP^{a} and | 2 times ULN with | Monitor liver tests twice weekly until ALP |
| GGT | GGT greater than | is less than or equal to 5 times ULN, then |
| | 2 times ULN | weekly until less than or equal to 2 times |
| | | ULN. |
| Increased | TB greater than | Withhold and monitor liver tests twice |
| bilirubin | ULN to less than | weekly. |
| | 2 times ULN or | If an alternate cause for increased |
| | DB greater than | bilirubin is confirmed and bilirubin is |
| | ULN and less | less than ULN within 4 weeks, resume |
| | than 1.5 times | at reduced dose. |
| | ULN | If bilirubin is not less than ULN |
| | | in 4 weeks, permanently |
| | | discontinue peddartinib. |
| | TB greater or | Permanently discontinue pexidartinib. |
| | equal to 2 times | Monitor liver tests twice weekly until |
| | ULN or | bilirubin is less than or equal to ULN. |
| | DB greater than | |
| | 1.5 times ULN | |

ALT = alanine aminotransferase; ALP = alkaline phosphatase; AST = aspartate aminotransferase; DB = direct bilirubin; GGT = gamma-glutamyl transferase; TB = total bilirubin; ULN = upper limit of normal.
^{a}Confirm ALP elevations as liver isozyme fraction.

Concomitant use of pexidartinib with strong CYP3A inhibitors or UGT inhibitors during should be avoided, in some embodiments. If concomitant use with a strong CYP3A inhibitor or UGT inhibitor cannot be avoided, the dose of pexidartinib should be reduced as described herein. If concomitant use of a strong CYP3A inhibitor or UGT inhibitor is discontinued, the dose of pexidartinib is increased (after 3 plasma half-lives of the strong CYP3A inhibitor or UGT inhibitor) to the dose that was used before starting the inhibitor.

In some embodiments, dose reductions for patients that are being treated concomitantly with CYP3A inhibitors or UGT inhibitors is conducted according to Table 3.

TABLE 3

| | | Administration of |
|---|---|---|
| Planned Total Daily | Modified Total Daily | Modified Total Daily |
| Dose of Pexidartinib | Dose of Pexidartinib | Dose of Pexidartinib |
| 800 mg | 400 mg | 200 mg twice daily |
| 600 mg* | 400 mg | 200 mg twice daily |
| 400 mg* | 200 mg | 200 mg once daily |

*Planned total daily dose refers to recommended dose reductions for pexidartinib for adverse reactions based on dosing recommendations in Table 2.

For purposes of clarification, the active ingredient of Turalio® is pexidartinib hydrochloride, and the active moiety is pexidartinib. A dosage of 200 mg of pexidartinib and EQ 200 mg of pexidartinib hydrochloride both have the same dosage of pexidartinib.

The concomitant use of proton pump inhibitors (PPI) while taking pexidartinib should be avoided. As an alternative to a PPI, pexidartinib may be administered with an antacid, for example 2 hours before or 2 hours after taking a locally-acting antacid, or if using a histamine 2 (H2)-receptor antagonist, at least 2 hours before or 10 hours after taking an H2-receptor antagonist. Patients having mild to severe renal impairment (creatinine clearance [CLcr] 15 to 89 mL/min estimated by Cockcroft-Gault using actual body weight) should be administered doses that are lower than the starting dose of 800 mg per day, e.g., by administering 200 mg in the morning and 400 mg in the evening.

### Risk Evaluation and Mitigation Programs

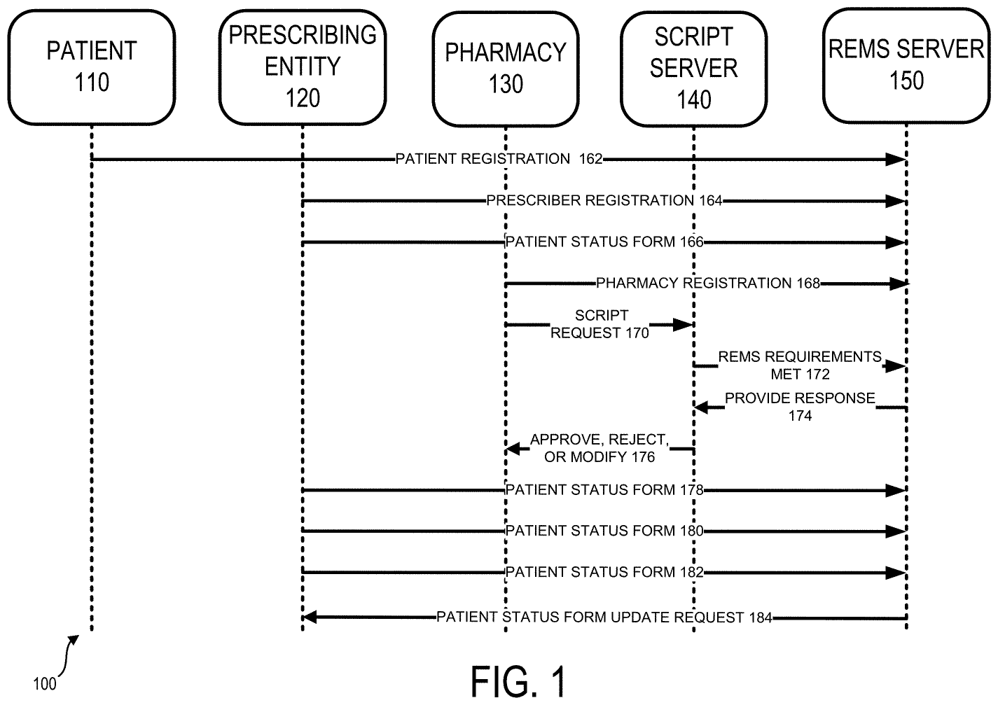

With reference to diagram 100 of FIG. 1, the current subject matter is directed to a computer-implemented risk evaluation and mitigation strategies (REMS) in connection with the delivery of pexidartinib, or a pharmaceutically acceptable salt thereof. The REMS as provided herein includes data exchange over one or more computing networks among various computing devices associated with a patient 110, a prescribing entity 120, a pharmacy 130, a script server 140, and a REMS server 150 which may include an associated database or series of databases. While FIG. 1 includes only a single computing device for illustration purposes, multiple computing devices (e.g., desktop computers, mobile devices, tablets, databases, servers, etc.) can be utilized for each entity involved with the REMS and, in particular, there may be dedicated databases within the REMS server 150 which separately store aspects of the current subject matter. Furthermore, some of the signaling (i.e., data messaging, etc.) illustrated in FIG. 1 may be combined or split into different messages depending on the desired configuration. Still further, the functionality provided by the script server 140 and the REM server 150 can be combined into a single computing device or cloud service.

The present disclosure also provides, in one embodiment, computer-implemented instructions and systems for implementing a risk evaluation and mitigation program. In accordance with the methods described herein, the delivery of pexidartinib, or a pharmaceutically acceptable salt thereof, to the patient may involve the following operations, at least some of which are implemented by one or more computing devices (e.g., computing devices 110-150). As a prelude to prescribing and dispensing pexidartinib, or a pharmaceutically acceptable salt thereof, to the patient, the prescriber and the pharmacy are registered in one or more appropriate computer readable storage media forming part of the REMS server 150 or otherwise accessible by the REMS server 150. If the prescriber is not registered in the computer readable storage medium, the prescriber will be ineligible to prescribe pexidartinib, or a pharmaceutically acceptable salt thereof. Similarly, if the pharmacy is not certified and registered in the computer readable storage medium, the pharmacy will be ineligible to dispense pexidartinib, or a pharmaceutically acceptable salt thereof. Further if the patient has not signed a form indicating that the patient has reviewed a patient guide and had the baseline liver function assessed, the patient may not be eligible to receive pexidartinib, or a pharmaceutically acceptable salt thereof, from the pharmacy. In some cases, a request that fails to meet these criteria will result in a message or other information to the prescriber, pharmacy or patient (as may be needed) with the requisite information/forms to register such entity.

In some embodiments, the prescriber or the pharmacy is registered in a specifically designed database (e.g., forming part of or otherwise connected to REMS server 150), together or separately, in non-transitory computer readable storage media. The term “database” refers generically to any collection of information that is structured, either as a traditional relation table or otherwise, to enable searches and updates. A database can be located locally on a computer device on which a processor receives a search query and conducts a search, or preferably remotely to the prescriber or pharmacy, through a computer network. In some embodiments, the databases are HIPAA-compliant.

The computer readable storage medium in which the pharmacies are registered may be the same as, or different from the computer readable storage medium in which the prescribers are registered. Once registered in the computer readable storage medium, the pharmacies may be eligible to dispense pexidartinib, or a pharmaceutically acceptable salt thereof, to patients who are in need of the drug.

Generally speaking, in order to become registered in the computer readable storage medium, the pharmacy and/or the prescriber may be required to comply with various aspects of the methods described herein including, for example, registering the patient (preferably also in a computer readable storage medium), ensuring that the patient complies with certain aspects of pexidartinib dosing, as well as other aspects of the present methods, such as scheduled liver testing. As with the registration of the prescriber in the computer readable storage medium, the registration of the pharmacy may be achieved by providing the pharmacy, for example, by mail, facsimile transmission, or on-line transmission, with a registration card or form, preferably together with appropriate educational materials concerning pexidartinib, or a pharmaceutically acceptable salt thereof.

In one embodiment, a method for administering pexidartinib, or a pharmaceutically acceptable salt thereof, while minimizing serious and potentially fatal liver injury, is provided. In some embodiments, the method entails registering a healthcare provider in a healthcare provider database. The registration can include the name, contact information, affiliation, specialty, and/or a reason for registration, in some embodiments. The registration, in some embodiments, requires that the healthcare provider has completed a knowledge assessment regarding pexidartinib. The requirement may be satisfied with the healthcare provider checking a box in a form presented in a graphical user interface provided by the system. Alternatively, the system is required to present a questionnaire to test the healthcare provider, or to check another database which hosts a listing of healthcare providers that have completed the assessment.

In some embodiments, the method requires that the healthcare provider assess a patient's baseline liver functions prior to making a prescription. In one embodiment, the computer-implemented system is configured to retrieve and display the baseline liver functions to the healthcare provider. In one embodiment, the system asks the healthcare provider to confirm that the assessment has been completed. In some embodiments, the system retrieves the test results and provide preliminary analysis for the healthcare provider.

Another requirement of the program, in one embodiment, is that each patient be registered in a database as well. Prior to such registration, in some embodiments, the system needs to confirm that the patient has reviewed a patient guide and had the baseline liver function assessed. The confirmation can be done by the patient (or a healthcare provider) checking a box, or presenting the patient guide to the patient, accompanied with sufficient time for the patient to review.

Upon confirmation of satisfaction of the above requirements, in some embodiments, the healthcare provider can then prescribe pexidartinib, or a pharmaceutically acceptable salt thereof, to the patient, which prescription can also be registered in the database. In some embodiments, the system can remind the healthcare provider and/or the patient for follow-up liver function assessment. Based on such re-assessments, the healthcare provider (optionally with the assistance from the computer system), the dosing of pexidartinib, or a pharmaceutically acceptable salt thereof, may be modified, as further disclosed below.

Yet another requirement, in some embodiments, of the system is that the pharmacy that dispenses the medication be also registered in the computer system. The pharmacy, in order to be registered, also needs to have completed a certification program regarding pexidartinib, or a pharmaceutically acceptable salt thereof. One of the requirements for the certification program is that the registered pharmacies only dispense pexidartinib, or a pharmaceutically acceptable salt thereof, to patients who are registered in the patient database.

Referring again to FIG. 1, various data exchange among the computing nodes 110-150 can be provided to effect the risk evaluation and mitigation program. It will be appreciated that the sequencing of the messaging can vary depending on various factors, including, the registration of the various entities. For example, patient registration data 162 may be transmitted from a computing device 110 associated with the patient over a network to the REMS server 150 for storage therein. This patient registration data 162 may encapsulate an identification of the patient, an identification of healthcare provider or provider associated with the patient, as well as additional information including consents and other acknowledgments (generated, for example, via one or more forms displayed in a graphical user interface, etc.). Similarly, the prescribing entity (e.g., healthcare provider, etc.) may transmit registration data 164 from a computing device 120 associated with such entity to the REMS server 150 for storage therein. The prescribing entity registration data 164 can encapsulate information identifying the prescribing entity, patient information, as well as other consents and other acknowledgments (generated, for example, via one or more forms displayed in a graphical user interface, etc.). Moreover, in some variations, the prescribing entity 120 can directly or indirectly (e.g., from a laboratory computing device, cloud storage, etc.) transmit a patient status form 166 that can, for example, characterize patient lab work data including a patient baseline test to the REMS server 150 which, in turn, can later be used to confirm eligibility of the patient for a script request or the like.

A computing device 130 associated with a pharmacy can transmit a request 170 to the script server 140 to fill (including refill) a prescription which comprises data encapsulating, for example, an identification of the pharmacy and the associated patient along with a requested dosage of pexidartinib, or a pharmaceutically acceptable salt thereof. In some cases, the request 170 can also include an identity of the prescribing entity. The script server 140 can then poll 172 the REMS server 150 to determine whether the script request 170 meets all of the requirements for the REMS program (as described elsewhere herein). The response of the REMS server 174 can then be used by the script server 140 to, for example, approve, reject or modify (e.g., change dosage and/or dosing frequency of pexidartinib, or a pharmaceutically acceptable salt thereof, and data characterizing same 176 can be transmitted back to the pharmacy 130. The pharmacist can then either fill the prescription as requested, modify the prescription, or reject the prescription as indicated in the response of the script server 140. Data signaling 170-176 can be repeated for each new script request.

The patient status form comprises an important aspect in the treatment of the patient and are required on a periodic basis. In some variations, multiple patient status forms 166, 178, 180, 182 are transmitted by the prescribing entity 120 to the REMS server 150. In some variations, the patient status forms have a limited validity window such that a prescription will be rejected if there is not a current/active patient status form. For example, a patient status form can be required on a monthly basis for months 1-3 of treatment, every three months for the remainder of the first year of treatment, and every six months thereafter. These windows can be measured from a benchmark date. The benchmark date can, for example, be a date when the patient first takes the medication, when the medication was first shipped to the patient, when the medication was first received by the patient after shipment, or when the medication is first given to the patient at a pharmacy. Information from the prescribing entity 120 is most critical when treatment commences and the patient status forms 166, 178, 180, and 182 can encapsulate information characterizing various tests including blood tests for the patient. For example, liver function tests can be conducted weekly for the first eight weeks of treatment, every two weeks for the next month, and every three months thereafter. The patient status forms 166, 178, 180, and 182 can, in some cases, encapsulate data characterizing an adverse event or a laboratory abnormality suggestion of serious and potentially fatal liver injury. Other information relating to dose reductions can be included which will then be incorporated into information sent to the script server 140 in connection with modifying a current prescription and the like. It will be appreciated that in FIG. 1, there can be intermediate requests to fill a prescription in between the various patient status forms 178-182.

In some cases, the REMS server 150 can transmit a patient status form update request 184 to the prescribing entity 120 when there is no longer an active patient status form for a patient. Further, in some variations, a grace period (e.g., 10 days, 20 days, etc.) can be specified and implemented such that REMS server 150 and/or the script server 140 causes the requested prescription to be filled even though the patient status form is not within a current validity window. Moreover, a patient 110 can be marked as inactive in the REMS server 150 if a patient status form is not received within a pre-defined time period after an expiration of a most recent validity window. Such time period need not correspond to a grace period, if any.

In some embodiments, if, after an initial pexidartinib treatment is administered, a patient's ALT and/or AST levels are determined to be greater than 3 to 5 times the corresponding ULN, pexidartinib treatment is suspended, and the patient undergoes liver function tests weekly till the patient's ALT and AST levels are less than 3 times the corresponding ULN. If a reduction in ALT and AST levels to less than 3 times the corresponding ULN is observed by the fourth week of suspended dosing, dosing of pexidartinib may be resumed at a lower dose compared to the starting dose of pexidartinib. If the patient's elevated ALT or AST levels are not decreased to less than 3 times the corresponding ULN by the fourth week, pexidartinib treatment is permanently discontinued for the patient.

In some embodiments, if, after an initial pexidartinib treatment is administered, a patient's ALT and/or AST levels are determined to be greater than 5 to 10 times the corresponding ULN, pexidartinib treatment is suspended, and the patient undergoes liver function tests twice a week till the patient's ALT and AST levels are less than 3 times the corresponding ULN. If a reduction in ALT and AST levels to less than 3 times the corresponding ULN is observed by the fourth week of suspended dosing, dosing of pexidartinib may be resumed at a lower dose compared to the starting dose of pexidartinib. If the patient's elevated ALT or AST levels are not decreased to less than 3 times the corresponding ULN by the fourth week, pexidartinib treatment is permanently discontinued for the patient.

In other embodiments, if, after an initial pexidartinib treatment is administered, a patient's ALT and/or AST levels are determined to be greater than 10 times the corresponding ULN, pexidartinib treatment is permanently discontinued for the patient. To avoid any fatal hepatotoxic events, the patient is tested twice a week till the patient's elevated ALT or AST levels are decreased to less than or equal to 5 times the corresponding ULN, then the patient is tested once a week till the patient's elevated ALT or AST levels are decreased to less than or equal to 3 times the corresponding ULN.

In some embodiments, if, after an initial pexidartinib treatment is administered, a patient's ALP level is determined to be greater than 2 times the corresponding ULN for ALP, and if a patient's GGT level is determined to be greater than 2 times the corresponding ULN for GGT, pexidartinib treatment is permanently discontinued for the patient. To avoid any fatal hepatotoxic events, the patient is tested twice a week till the patient's elevated ALP levels are decreased to less than or equal to 5 times the corresponding ULN, then the patient is tested once a week till the patient's elevated ALP levels are decreased to less than or equal to 2 times the corresponding ULN.

In some embodiments, if, after an initial pexidartinib treatment is administered, a patient's TB is determined to be greater than the corresponding ULN for TB to less than 2 times the corresponding ULN for TB, pexidartinib treatment is suspended, and the patient undergoes liver function tests weekly till the patient's TB levels are less than the corresponding ULN. If a reduction in TB levels to less than the corresponding ULN is observed by the fourth week of suspended dosing, dosing of pexidartinib may be resumed at a lower dose compared to the starting dose of pexidartinib. If the patient's elevated TB levels are not decreased to less than the corresponding ULN by the fourth week, pexidartinib treatment is permanently discontinued for the patient.

In some embodiments, if, after an initial pexidartinib treatment is administered, a patient's DB is determined to be greater than the corresponding ULN for DB and less than 1.5 times the corresponding ULN for DB, pexidartinib treatment is suspended, and the patient undergoes liver function tests weekly till the patient's DB levels are less than the corresponding ULN. If a reduction in DB levels to less than the corresponding ULN is observed by the fourth week of suspended dosing, dosing of pexidartinib may be resumed at a lower dose compared to the starting dose of pexidartinib. If the patient's elevated DB levels are not decreased to less than the corresponding ULN by the fourth week, pexidartinib treatment is permanently discontinued for the patient.

In some embodiments, if, after an initial pexidartinib treatment is administered, a patient's TB is determined to be greater than or equal to 2 times the corresponding ULN for TB, pexidartinib treatment is permanently discontinued. To avoid any fatal hepatotoxic events, the patient is tested twice a week till the patient's elevated TB levels are decreased to less than or equal to the corresponding ULN.

In some embodiments, if, after an initial pexidartinib treatment is administered, a patient's DB is determined to be greater than the corresponding ULN for DB and less than 1.5 times the corresponding ULN for DB, pexidartinib treatment is permanently discontinued. To avoid any fatal hepatotoxic events, the patient is tested twice a week till the patient's elevated DB levels are decreased to less than or equal to the corresponding ULN.

In some embodiments, where a patient suffers from mild to severe renal impairment (creatinine clearance [CLcr] 15 to 89 mL/min estimated by Cockcroft-Gault using actual body weight) pexidartinib treatment may be initiated at a reduced dose (e.g., 200 mg in the morning and 400 mg in the evening) followed by liver function measurements as described herein. The patients daily dosing of pexidartinib may be adjusted (reduced or discontinued) based on liver function measurements.

Accordingly, in some or any of the embodiments described herein, periodic monitoring of a patient's liver function after initiation of pexidartinib therapy (e.g., by measuring ALT and/or AST level, and/or by measuring ALP and GGT levels, and/or by measuring increase in bilirubin) reduces the occurrence of, and mitigates the risk of, fatal hepatotoxicities. Periodic liver function measurements may be undertaken at any suitable interval of time, e.g., each day, every other day, twice a week, once a week, every two weeks, once a month, every six months, once a year, or any other suitable frequency. A co-administered medication may also impact liver function. Accordingly, in some embodiments, administration of pexidartinib with medications that affect liver function should be avoided. Examples of medications that interact with pexidartinib and that may increase the potential of liver injury from pexidartinib include, and are not limited to, CYP3A inhibitors, UDP-glucuronosyltransferase (UGT) inhibitors, and the like. In other embodiments, pexidartinib should be administered at reduced doses if co-administration of medications that may impact liver function cannot be avoided.

In a particular embodiment, the methods described herein comprise administering to the subject an initial daily dose of about 800 mg equivalent of pexidartinib per day described herein and adjusting the dose to avoid serious and potentially fatal liver injury according to the methods described herein. The dosage can be decreased from about 800 mg equivalent of pexidartinib per day to about 600 mg equivalent of pexidartinib per day, about 400 mg equivalent of pexidartinib per day, or about 200 mg equivalent of pexidartinib per day. In some embodiments, the dosing is permanently discontinued. In such embodiments, the dose is adjusted based on liver function assessments as described herein.

In accord with the methods described herein, in some embodiments, a dose reduction of pexidartinib, or a pharmaceutically acceptable salt thereof, is carried out according to Table 1. Starting from a total daily dose of 800 mg equivalent of pexidartinib daily, the first dose reduction comprises reducing the dose of pexidartinib, or a pharmaceutically acceptable salt thereof, to 600 mg equivalent of pexidartinib daily. The second dose reduction comprises reducing the dose of pexidartinib, or a pharmaceutically acceptable salt thereof, to 400 mg equivalent of pexidartinib daily.

In some embodiments, each of the initial prescription, the liver function testing, re-assessment, adjustment or termination of doses, is recorded in the computer system.

In some embodiments, each of the initial prescription, the baseline liver function testing, liver function testing if/when an adverse event occurs, termination of doses, is recorded in the computer system.

In some or any of the embodiments described herein, the methods described herein further comprises the employment of UBC software to access and process any of said data.

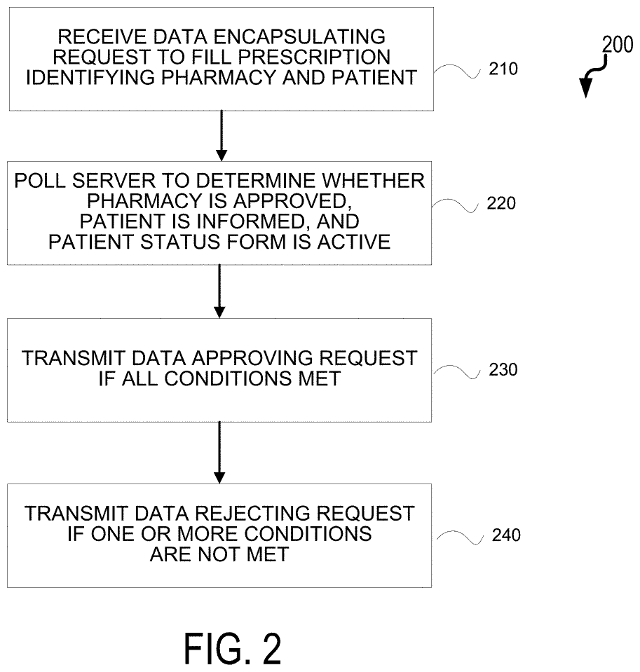

FIG. 2 is a diagram 200 in which, at 210, at least one computing device receives data encapsulating a request to fill a prescription for pexidartinib, or a pharmaceutically acceptable salt thereof, for a patient. The request can specify information relevant to the prescription including a pharmacy initiating the request and an identification of the patient. In response to the request, at 220, a server is polled with the pharmacy identification and the patient identification to determine whether (i) the pharmacy is registered and pre-approved to fill prescriptions for pexidartinib, or a pharmaceutically acceptable salt thereof, (ii) the patient executed a consent form or other acknowledgement associated with the administration of pexidartinib, or a pharmaceutically acceptable salt thereof, and (iii) the patient has a patient status form prepared by a prescribing entity transmitted or prepared within a corresponding validity window thereby making such patient status form active. In some variations, the validity windows increases in length of days as treatment of the patient with pexidartinib, or a pharmaceutically acceptable salt thereof, progresses. Data responsive to the request can be transmitted at 230 approving filling of the prescription by the pharmacy if it is determined that the pharmacy is registered and pre-approved to fill prescriptions for pexidartinib, or a pharmaceutically acceptable salt thereof, the patient has executed the consent form or other acknowledgment, and the patient has an active patient status form. Alternatively, at 240, data responsive to the request can be transmitted rejecting filling of the prescription by the pharmacy if it is determined (a) that the pharmacy is not registered and pre-approved to fill prescriptions for pexidartinib, or a pharmaceutically acceptable salt thereof, (b) the patient has executed the form, or (c) the patient does not have an active patient status form.

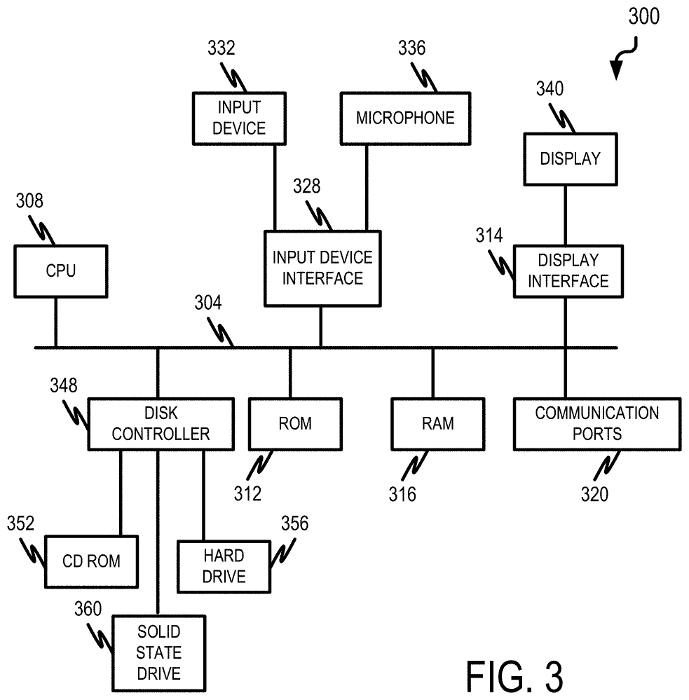

FIG. 3 is a diagram 300 illustrating a sample computing device architecture for implementing various aspects described herein. A bus 304 can serve as the information highway interconnecting the other illustrated components of the hardware. A processing system 308 labeled CPU (central processing unit) (e.g., one or more computer processors/data processors at a given computer or at multiple computers), can perform calculations and logic operations required to execute a program. A non-transitory processor-readable storage medium, such as read only memory (ROM) 312 and random access memory (RAM) 316, can be in communication with the processing system 308 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 348 can interface with one or more optional disk drives to the system bus 304. These disk drives can be external or internal CD-ROM, CD-R, CD-RW or DVD 352, or solid state drives such as 352, or external or internal hard drives 356. As indicated previously, these various disk drives 352, 356, 360 and disk controllers are optional devices. The system bus 304 can also include at least one communication port 320 to allow for communication with external devices either physically connected to the computing system or available externally through a wired or wireless network. In some cases, the at least one communication port 320 includes or otherwise comprises a network interface.

To provide for interaction with a user, the subject matter described herein can be implemented on a computing device having a display device 340 (e.g., a CRT (cathode ray tube) or LCD (liquid crystal display) monitor) for displaying information obtained from the bus 304 via a display interface 314 to the user and an input device 332 such as keyboard and/or a pointing device (e.g., a mouse or a trackball) and/or a touchscreen by which the user can provide input to the computer. Other kinds of input devices 332 can be used to provide for interaction with a user as well; for example, feedback provided to the user can be any form of sensory feedback (e.g., visual feedback, auditory feedback by way of a microphone 336, or tactile feedback); and input from the user can be received in any form, including acoustic, speech, or tactile input. The input device 332 and the microphone 336 can be coupled to and convey information via the bus 304 by way of an input device interface 328. Other computing devices, such as dedicated servers, can omit one or more of the display 340 and display interface 314, the input device 332, the microphone 336, and input device interface 328.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

Unless otherwise defined, all technical and scientific terms used herein have the same meaning as commonly understood by one of ordinary skill in the art to which this subject matter belongs.

The current subject matter may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed subject matter.

All publications, patent applications, patents, and other references mentioned herein are expressly incorporated by reference in their entirety, to the same extent as if each were incorporated by reference individually. In case of conflict, the present specification, including definitions, will control.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method comprising:
receiving, by at least one computing device, data encapsulating a request to fill a prescription for pexidartinib, or a pharmaceutically acceptable salt thereof, for a patient, the request specifying a pharmacy initiating the request and including an identification of the patient;
polling a server with the pharmacy identification and the patient identification to determine whether
(i) the pharmacy is registered and pre-approved to fill prescriptions for pexidartinib, or a pharmaceutically acceptable salt thereof;
(ii) the patient executed a consent form or other acknowledgement associated with the administration of pexidartinib, or a pharmaceutically acceptable salt thereof, and
(iii) the patient has a patient status form prepared by a prescribing entity transmitted or prepared within a corresponding validity window thereby making such patient status form active, the validity windows increasing in length of days as treatment of the patient with pexidartinib, or a pharmaceutically acceptable salt thereof, progresses;
transmitting data responsive to the request approving filling of the prescription by the pharmacy if it is determined that the pharmacy is registered and pre-approved to fill prescriptions for pexidartinib, or a pharmaceutically acceptable salt thereof, the patient has executed the consent form or other acknowledgment, and the patient has an active patient status form; and
transmitting data responsive to the request rejecting filling of the prescription by the pharmacy if it is determined (a) that the pharmacy is not registered and pre-approved to fill prescriptions for pexidartinib, or a pharmaceutically acceptable salt thereof, (b) the patient has executed the form, or (c) the patient does not have an active patient status form.

2. The method of claim 1, wherein the patient status form encapsulates data regarding at least one liver function test performed by the patient prior to and/or after commencement of treatment.

3. The method of claim 2, wherein the liver function tests are conducted on a frequency of weekly for the first eight weeks of treatment, every two weeks during a third month of treatment, every three months until a first year anniversary of treatment and every six months thereafter.

4. The method of claim 1, wherein the validity windows has a length equal to one month for months 1 to 3 from a benchmark date, the validity windows have a length equal to three months for months 3 to 12 from the benchmark date, and the validity windows have a length equal to six months after month 12 from the benchmark date.

5. The method of claim 4, wherein the benchmark date is a date on which the prescription for pexidartinib, or a pharmaceutically acceptable salt thereof, was first shipped to the patient.

6. The method of claim 4, wherein the benchmark date is a date on which the prescription for pexidartinib, or a pharmaceutically acceptable salt thereof, was first received by the patient by mail.

7. The method of claim 4, wherein the benchmark date is a date on which the prescription for pexidartinib, or a pharmaceutically acceptable salt thereof, was first received by the patient at pharmacy.

8. The method of claim 1 further comprising:
determining that a most recent patient status form for the patient is outside a corresponding validity window but within a pre-specified grace period;
transmitting data responsive to the request approving filling of the prescription by the pharmacy if it is determined that the pharmacy is registered and pre-approved to fill prescriptions for pexidartinib, or a pharmaceutically acceptable salt thereof, and the patient has executed the consent form or other acknowledgment.

9. The method of claim 8, wherein the grace period is twenty (20) days.

10. The method of claim 8 further comprising:
determining that a most recent patient status form for the patient is outside a corresponding validity window and outside the pre-specified grace period.

11. The method of claim 10 further comprising:
marking the patient as inactive at the server in response to the determination that a most recent patient status form for the patient is outside the corresponding validity window and outside the pre-specified grace period.

12. The method of claim 10 further comprising:
transmitting data to the prescribing entity indicating that the patient is overdue for a patient status form and requesting the prescribing entity to submit an updated patient status form.

13. The method of claim 1 further comprising:
polling the server with an identification of a prescribing entity; and
wherein the request to fill the prescription is approved if the server indicates that the prescribing entity is authorized to fill the prescription and the request to fill the prescription is rejected if the server indicates that the prescribing entity is not authorized to fill the prescription.

14. The method of claim 1 further comprising:
transmitting data responsive to the request modifying a dosage and/or dosage frequency based on the patient status form.

15. The method of claim 1, wherein the server is a risk evaluation and mitigation strategies (REMS) server having an associated database storing patient, prescribing entity, and pharmacy information.

16. The method of claim 15 further comprising:
transmitting data to the REMS server to register healthcare providers in the database who have completed a knowledge assessment regarding pexidartinib.

17. The method of claim 15 further comprising:
transmitting lab work data characterizing liver function of the patient during treatment with pexidartinib, or a pharmaceutically acceptable salt thereof, to the REMS server for storage in the database.

18. The method of claim 15 further comprising:
transmitting data to the REMS server to register patients in the database.

19. The method of claim 18, wherein the acknowledgements from the patients comprises an acknowledgment that a patient guide has been reviewed.

20. The method of claim 16 further comprising:
transmitting data to the REMS server to register pharmacies in the database who have completed a certification program regarding pexidartinib, or a pharmaceutically acceptable salt thereof.

21. The method of claim 20 further comprising:
transmitting a consent form or other acknowledgment to patient requesting a prescription who is not registered in the patient database in response to such request.

22. The method claim 1 further comprising:
a) administering to the patient a dose of 400 mg equivalent of pexidartinib twice daily;
b) monitoring the patient by measuring the patient's liver function; and
c) reducing the dose or permanently discontinuing administration based on the patient's liver function.

23. The method of claim 22, wherein the patient is being treated for symptomatic tenosynovial giant cell tumor (TGCT).

24. The method of claim 22, wherein the measuring of the patient's liver function comprises measuring levels of alanine transaminase (ALT), aspartate transaminase (AST), alkaline phosphatase (ALP), gamma-glutamyl transferase (GGT), total bilirubin (TB) and direct bilirubin (DB) or a combination thereof.

25. The method of claim 24, wherein if the ALT or AST level is greater than 3 to 5 times the corresponding upper limit of normal (ULN), then
the administration of pexidartinib, or a pharmaceutically acceptable salt thereof, is withheld and the liver function measurements are conducted weekly; and
i) if the AST and ALT levels are less than or equal to 3 times the corresponding ULN within 4 weeks, the administration of pexidartinib, or a pharmaceutically acceptable salt thereof, is resumed at a reduced dose; or
ii) if the AST or ALT level is not less than or equal to 3 times the corresponding ULN in 4 weeks, the administration of pexidartinib, or a pharmaceutically acceptable salt thereof, is permanently discontinued.

26. The method of claim 24, wherein if the ALT or AST level is greater than 5 to 10 times the corresponding upper limit of normal (ULN), then
the administration of pexidartinib, or a pharmaceutically acceptable salt thereof, is withheld and the liver function measurements are conducted twice a week; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times the corresponding ULN in 4 weeks, the administration of pexidartinib, or a pharmaceutically acceptable salt thereof, is permanently discontinued.

27. The method of claim 24, wherein if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then
the liver function measurements are conducted weekly until the AST or ALT levels are less than or equal to 3 times the corresponding ULN.

28. The method of claim 24, wherein if the ALP level is greater than 2 times the corresponding ULN and the GGT is greater than 2 times the corresponding ULN, then
a) the administration of pexidartinib, or a pharmaceutically acceptable salt thereof, is permanently discontinued; and
b) the liver function measurements are conducted twice a week until the ALP level is less than or equal to 5 times the corresponding ULN, and then
the liver function measurements are conducted weekly until the ALP level is less than or equal to 2 times the corresponding ULN.

29. The method of claim 24, wherein the measuring of the patient's liver function comprises measuring levels of total bilirubin (TB), direct bilirubin (DB), or a combination thereof.

30. The method of claim 29, wherein if the TB level is greater than the corresponding ULN and less than 2 times the corresponding ULN or the DB level is greater than the corresponding ULN and less than 1.5 times the corresponding ULN, then
the administration of pexidartinib, or a pharmaceutically acceptable salt thereof, is withheld and the liver function measurements are conducted twice a week; and
a) if an alternate cause for the increased bilirubin is confirmed and the TB or DB level is less than the corresponding ULN within 4 weeks, the administration of pexidartinib, or a pharmaceutically acceptable salt thereof, is resumed at a reduced dose; or
b) if the TB or DB level is not less than corresponding ULN in 4 weeks, the administration of pexidartinib, or a pharmaceutically acceptable salt thereof, is permanently discontinued.

31. The method of claim 29, wherein if the TB level is greater than or equal to 2 times the corresponding ULN or the DB level is greater than 1.5 times the corresponding ULN, then
a) the administration of pexidartinib, or a pharmaceutically acceptable salt thereof, is permanently discontinued; and
b) the liver function measurements are conducted twice a week until the TB or DB level is less than or equal to the corresponding ULN.

32. The method of claim 22, wherein pexidartinib, or a pharmaceutically acceptable salt thereof, is not co-administered with a cytochrome P450 3A (CYP3A) inhibitor, a uridine diphosphate-glucuronosyltransferase (UGT) inhibitor, or a combination thereof.

33. The method of claim 22, wherein pexidartinib, or a pharmaceutically acceptable salt thereof, is administered at a reduced dose if co-administered with a CYP3A inhibitor, a UDP-glucuronosyltransferase (UGT) inhibitor, or a combination thereof.

34. The method of claim 22, wherein pexidartinib, or a pharmaceutically acceptable salt thereof, is not co-administered with a proton pump inhibitor.

35. The method of claim 22, wherein pexidartinib, or a pharmaceutically acceptable salt thereof, is administered at a reduced dose in the patient, wherein the patient has mild to severe renal impairment as defined by creatinine clearance [CLcr] 15 to 89 mL/min estimated by Cockcroft-Gault using actual body weight.

36. The method of claim 25, wherein a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

37. The method of claim 1 further comprising:
a) registering one or more healthcare providers in a healthcare provider database in a non-transitory computer readable storage medium, wherein the one or more healthcare providers have completed a knowledge assessment regarding pexidartinib;
b) requiring the one or more healthcare providers to assess the patient's baseline liver functions;
c) registering the patient in a database, wherein the patients has reviewed a patient guide and has had a baseline liver function assessed; and
d) during treatment, re-assessing the patient's liver functions and modifying a dose of pexidartinib, or a pharmaceutically acceptable salt thereof, as needed.

38. The method of claim 37, further comprising registering one or more pharmacies which have completed a certification program regarding pexidartinib, or a pharmaceutically acceptable salt thereof, in a pharmacy database, and wherein the one or more pharmacies only dispense pexidartinib, or a pharmaceutically acceptable salt thereof, to patients who are registered in the patient database.

39. The method of claim 37, wherein the assessing of the patient's baseline liver function comprises measuring levels of alanine transaminase (ALT), aspartate transaminase (AST), alkaline phosphatase (ALP), gamma-glutamyl transferase (GGT), total bilirubin (TB) and direct bilirubin (DB) or a combination thereof.

40. The method of claim 39, wherein if the ALT or AST level is greater than 3 to 5 times the corresponding upper limit of normal (ULN), then
the administration of pexidartinib, or a pharmaceutically acceptable salt thereof, is withheld and the liver function measurements are conducted weekly; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times the corresponding ULN in 4 weeks, the administration of pexidartinib, or a pharmaceutically acceptable salt thereof, is permanently discontinued.

41. The method of claim 39, wherein if the ALT or AST level is greater than 5 to 10 times the corresponding upper limit of normal (ULN), then
the administration of pexidartinib, or a pharmaceutically acceptable salt thereof, is withheld and the liver function measurements are conducted twice a week; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times ULN in 4 weeks, the administration of pexidartinib, or a pharmaceutically acceptable salt thereof, is permanently discontinued.

42. The method of claim 39, wherein if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then
the liver function measurements are conducted weekly until the AST or ALT levels are less than or equal to 3 times the corresponding ULN.

43. The method of claim 39, wherein if the ALP level is greater than 2 times the corresponding ULN with the GGT greater than 2 times the corresponding ULN, then
a) the administration of pexidartinib, or a pharmaceutically acceptable salt thereof, is permanently discontinued; and
b) the liver function measurements are conducted twice a week until ALP level is less than or equal to 5 times the corresponding ULN and then
the liver function measurements are conducted weekly until ALP level is are less than or equal to 2 times the corresponding ULN.

44. The method of claim 37, wherein the assessing of the patient's liver function comprises measuring levels of total bilirubin (TB), direct bilirubin (DB), or a combination thereof.

45. The method of claim 44, wherein if TB is greater than the corresponding ULN to less than 2 times the corresponding ULN or DB is greater than the corresponding ULN and less than 1.5 times the corresponding ULN, then
a) the administration of pexidartinib, or a pharmaceutically acceptable salt thereof, is withheld and liver function measurements are conducted twice a week; and
b) if an alternate cause for the increased bilirubin is confirmed and the TB or DB level is less than the corresponding ULN within 4 weeks, the administration of pexidartinib, or a pharmaceutically acceptable salt thereof, is resumed at a reduced dose; or
c) if the TB or DB level is not less than the corresponding ULN in 4 weeks, the administration of pexidartinib, or a pharmaceutically acceptable salt thereof, is permanently discontinued.

46. The method of claim 44, wherein if the TB level is greater or equal to 2 times the corresponding ULN or DB is greater than 1.5 times the corresponding ULN, then
a) the administration of pexidartinib, or a pharmaceutically acceptable salt thereof, is permanently discontinued; and
b) the liver function measurements are conducted twice a week until the TB or DB levels is less than or equal to the corresponding ULN.

47. The method of claim 37, wherein pexidartinib, or a pharmaceutically acceptable salt thereof, is not co-administered with a CYP3A inhibitor, a UDP-glucuronosyltransferase (UGT) inhibitor, or a combination thereof.

48. The method of claim 37, wherein pexidartinib, or a pharmaceutically acceptable salt thereof, is administered at a reduced dose if co-administered with a CYP3A inhibitor, a UDP-glucuronosyltransferase (UGT) inhibitor, or a combination thereof.

49. The method of claim 37, wherein pexidartinib, or a pharmaceutically acceptable salt thereof, is not co-administered with a proton pump inhibitor.

50. The method of claim 37, wherein pexidartinib, or a pharmaceutically acceptable salt thereof, is administered at a reduced dose in the patient, wherein the patient has mild to severe renal impairment as defined by creatinine clearance [CLcr] 15 to 89 mL/min estimated by Cockcroft-Gault using actual body weight.

51. The method of claim 40, wherein a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

52. The method of claim 1, wherein the patient having symptomatic tenosynovial giant cell tumor (TGCT), the method further comprising:
a) administering to the patient a dose of 400 mg equivalent of pexidartinib twice daily;
b) monitoring the patient by measuring the patient's liver function; and
c) reducing the dose or permanently discontinuing administration based on the patient's liver function.

53. The method of claim 52, wherein the measuring of the patient's liver function comprises measuring levels of alanine transaminase (ALT), aspartate transaminase (AST), alkaline phosphatase (ALP), gamma-glutamyl transferase (GGT), total bilirubin (TB) and direct bilirubin (DB) or a combination thereof.

54. The method of claim 53, wherein if the ALT or AST level is greater than 3 to 5 times the corresponding upper limit of normal (ULN), then
the administration of pexidartinib, or a pharmaceutically acceptable salt thereof, is withheld and the liver function measurements are conducted weekly; and
i) if the AST and ALT levels are less than or equal to 3 times the corresponding ULN within 4 weeks, the administration of pexidartinib, or a pharmaceutically acceptable salt thereof, is resumed at a reduced dose; or
ii) if the AST or ALT level is not less than or equal to 3 times the corresponding ULN in 4 weeks, the administration of pexidartinib, or a pharmaceutically acceptable salt thereof, is permanently discontinued.

55. The method of claim 53, wherein if the ALT or AST level is greater than 5 to 10 times the corresponding upper limit of normal (ULN), then
the administration of pexidartinib, or a pharmaceutically acceptable salt thereof, is withheld and the liver function measurements are conducted twice a week; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times the corresponding ULN in 4 weeks, the administration of pexidartinib, or a pharmaceutically acceptable salt thereof, is permanently discontinued.

56. The method of claim 53, wherein if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then
the liver function measurements are conducted weekly until the AST or ALT levels are less than or equal to 3 times the corresponding ULN.

57. The method of claim 53, wherein if the ALP level is greater than 2 times the corresponding ULN and the GGT is greater than 2 times the corresponding ULN, then
a) the administration of pexidartinib, or a pharmaceutically acceptable salt thereof, is permanently discontinued; and
b) the liver function measurements are conducted twice a week until the ALP level is less than or equal to 5 times the corresponding ULN, and then
the liver function measurements are conducted weekly until the ALP level is are less than or equal to 2 times the corresponding ULN.

58. The method of claim 52, wherein the measuring the patient's liver function comprises measuring levels of total bilirubin (TB), direct bilirubin (DB), or a combination thereof.

59. The method of claim 58, wherein if the TB level is greater than the corresponding ULN and less than 2 times the corresponding ULN or the DB level is greater than the corresponding ULN and less than 1.5 times the corresponding ULN, then
the administration of pexidartinib, or a pharmaceutically acceptable salt thereof, is withheld and the liver function measurements are conducted twice a week; and
a) if an alternate cause for the increased bilirubin is confirmed and the TB or DB level is less than the corresponding ULN within 4 weeks, the administration of pexidartinib, or a pharmaceutically acceptable salt thereof, is resumed at a reduced dose; or
b) if the TB or DB level is not less than corresponding ULN in 4 weeks, the administration of pexidartinib, or a pharmaceutically acceptable salt thereof, is permanently discontinued.

60. The method of claim 58, wherein if the TB level is greater than or equal to 2 times the corresponding ULN or the DB level is greater than 1.5 times the corresponding ULN, then
a) the administration of pexidartinib, or a pharmaceutically acceptable salt thereof, is permanently discontinued; and
b) the liver function measurements are conducted twice a week until the TB or DB level is less than or equal to the corresponding ULN.

61. The method of claim 53, wherein pexidartinib, or a pharmaceutically acceptable salt thereof, is not co-administered with a cytochrome P450 3A (CYP3A) inhibitor, a uridine diphosphate-glucuronosyltransferase (UGT) inhibitor, or a combination thereof.

62. The method of claim 53, wherein pexidartinib, or a pharmaceutically acceptable salt thereof, is administered at a reduced dose if co-administered with a CYP3A inhibitor, a UDP-glucuronosyltransferase (UGT) inhibitor, or a combination thereof.

63. The method of claim 53, wherein pexidartinib, or a pharmaceutically acceptable salt thereof, is not co-administered with a proton pump inhibitor.

64. The method of claim 53, wherein pexidartinib, or a pharmaceutically acceptable salt thereof, is administered at a reduced dose in a patient, wherein the patient has mild to severe renal impairment as defined by creatinine clearance [CLcr] 15 to 89 mL/min estimated by Cockcroft-Gault using actual body weight.

65. The method of claim 54, wherein a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

66. A method for treating a patient with pexidartinib, or a pharmaceutically acceptable salt thereof, while minimizing a risk of serious and potentially fatal liver injury, wherein the patient having symptomatic tenosynovial giant cell tumor (TGCT), the method comprising:
a) administering to the patient a dose of 400 mg equivalent of pexidartinib twice daily;
b) registering one or more healthcare providers in a healthcare provider database in a non-transitory computer readable storage medium, wherein the one or more healthcare providers have completed a knowledge assessment regarding pexidartinib;
c) requiring the one or more healthcare providers to assess the patient's baseline liver functions;
d) registering the patient in a database, wherein the patient has reviewed a patient guide and has had a baseline liver function assessed; and
e) during treatment, re-assessing a patient's liver function and reducing the dose or permanently discontinuing administration based on the patient's liver function.

67. The method of claim 66, further comprising registering one or more pharmacies which have completed a certification program regarding pexidartinib, or a pharmaceutically acceptable salt thereof, in a pharmacy database, and wherein the pharmacies only dispense pexidartinib, or a pharmaceutically acceptable salt thereof, to patients who are registered in the patient database.

68. The method of claim 66, wherein the assessing of the patient's baseline liver function comprises measuring levels of alanine transaminase (ALT), aspartate transaminase (AST), alkaline phosphatase (ALP), gamma-glutamyl transferase (GGT), total bilirubin (TB) and direct bilirubin (DB) or a combination thereof.

69. The method of claim 68, wherein if the ALT or AST level is greater than 3 to 5 times the corresponding upper limit of normal (ULN), then
the administration of pexidartinib, or a pharmaceutically acceptable salt thereof, is withheld and the liver function measurements are conducted weekly; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times the corresponding ULN in 4 weeks, the administration of pexidartinib, or a pharmaceutically acceptable salt thereof, is permanently discontinued.

70. The method of claim 68, wherein if the ALT or AST level is greater than 5 to 10 times the corresponding upper limit of normal (ULN), then
the administration of pexidartinib, or a pharmaceutically acceptable salt thereof, is withheld and the liver function measurements are conducted twice a week; and
a) if the AST and ALT levels are less than or equal to 3 times the corresponding ULN within 4 weeks, the administration of pexidartinib, or a pharmaceutically acceptable salt thereof, is resumed at a reduced dose; or
b) if the AST or ALT level is not less than or equal to 3 times ULN in 4 weeks, the administration of pexidartinib, or a pharmaceutically acceptable salt thereof, is permanently discontinued.

71. The method of claim 68, wherein if the ALT or AST level is greater than 10 times the corresponding upper limit of normal (ULN), then
a) the administration of pexidartinib, or a pharmaceutically acceptable salt thereof, is permanently discontinued; and
b) the liver function measurements are conducted twice a week until the AST or ALT levels are less than or equal to 5 times the corresponding ULN, and then
the liver function measurements are conducted weekly until the AST or ALT levels are less than or equal to 3 times the corresponding ULN.

72. The method of claim 68, wherein if the ALP level is greater than 2 times the corresponding ULN with the GGT greater than 2 times the corresponding ULN, then
a) the administration of pexidartinib, or a pharmaceutically acceptable salt thereof, is permanently discontinued; and
b) the liver function measurements are conducted twice a week until ALP level is less than or equal to 5 times the corresponding ULN, and then
the liver function measurements are conducted weekly until ALP level is are less than or equal to 2 times the corresponding ULN.

73. The method of claim 66, wherein the assessing of the patient's liver function comprises measuring levels of total bilirubin (TB), direct bilirubin (DB), or a combination thereof.

74. The method of claim 63, wherein if TB is greater than the corresponding ULN to less than 2 times the corresponding ULN or DB is greater than the corresponding ULN and less than 1.5 times the corresponding ULN, then
a) the administration of pexidartinib, or a pharmaceutically acceptable salt thereof, is withheld and liver function measurements are conducted twice a week; and
b) if an alternate cause for the increased bilirubin is confirmed and the TB or DB level is less than the corresponding ULN within 4 weeks, the administration of pexidartinib, or a pharmaceutically acceptable salt thereof, is resumed at a reduced dose; or
c) if the TB or DB level is not less than the corresponding ULN in 4 weeks, the administration of pexidartinib, or a pharmaceutically acceptable salt thereof, is permanently discontinued.

75. The method of claim 63, wherein if the TB level is greater or equal to 2 times the corresponding ULN or DB is greater than 1.5 times the corresponding ULN, then
a) the administration of pexidartinib, or a pharmaceutically acceptable salt thereof, is permanently discontinued; and
b) the liver function measurements are conducted twice a week until the TB or DB levels is less than or equal to the corresponding ULN.

76. The method of claim 66, wherein pexidartinib, or a pharmaceutically acceptable salt thereof, is not co-administered with a CYP3A inhibitor, a UDP-glucuronosyltransferase (UGT) inhibitor, or a combination thereof.

77. The method of claim 66, wherein pexidartinib, or a pharmaceutically acceptable salt thereof, is administered at a reduced dose if co-administered with a CYP3A inhibitor, a UDP-glucuronosyltransferase (UGT) inhibitor, or a combination thereof.

78. The method of claim 66, wherein pexidartinib, or a pharmaceutically acceptable salt thereof, is not co-administered with a proton pump inhibitor.

79. The method of claim 66, wherein pexidartinib, or a pharmaceutically acceptable salt thereof, is administered at a reduced dose in the patient, wherein the patient has mild to severe renal impairment as defined by creatinine clearance [CLcr] 15 to 89 mL/min estimated by Cockcroft-Gault using actual body weight.

80. The method of claim 69, wherein a reduced dose of pexidartinib, or a pharmaceutically acceptable salt thereof, is a change in dose from 800 mg equivalent of pexidartinib daily to 600 mg equivalent of pexidartinib daily or 400 mg equivalent of pexidartinib daily.

81. A system comprising:
at least one data processor; and
memory storing instructions which, when executed by the at least one data processor, implement a method of claim 1.

82. A non-transitory computer program product comprising instructions which, when executed by at least one computing device, result in a method of claim 1.
